SEQUENCE LISTING (32 sequences)

SEQ ID NO: 1 - DNA, 807 bp, Caldilinea aerophila
  source 1..807: mol_type = genomic DNA
atgaacgatcccgttctggaagccagaccgattttggagagatttcgcctcgacggccgcgtggcgctggtgaccggcggtgggcagggcatcggccgcgccttcgcccatgcgctgtgcgaggcaggcgccgcagtggccgtcgtcgatctgcgcctggacctggccgaggaagtggcgcatgagctggtaaacaagcagatcgacgccatcgcaattcaggccgatgtgacgaagcccgatcaggtgcagacgatggttgatgccattctgtccaggtgggggacgttgacgatcggtgtcaacaacgccggcatcggcttgtgggcggatgccgaaagcatggcatacacagactggctgcgcgtgatcgatattgatctgaactcggttttcctctgcgcccaggctgaagcgagggtcatgctgccggccggctacggcaagatcatcaacacggcgtcgatgtccggccatatcagcaacacgccgcaaaatcaggctgcttacaacacggccaaggctggcgtgatccatctgacgcgcagccttgccgcggaatgggccaagcggggagtgcgcgtcaacagcatcagccccggctatacgcgcaccaaattggttgacgacctgctcgccacgccgatcggacagacgatgttgcccacctggatgggcatgacgcccatgggacgaatggccgaggtcaccgatctgcaaggcgccgtcgtctacctggcgtcgtcggcgtccgactatatgaccggccatgacatggtgattgacggcggctattgttgctggtaa

SEQ ID NO: 2 - protein, 268 aa, Caldilinea aerophila
MNDPVLEARPILERFRLDGRVALVTGGGQGIGRAFAHALCEAGAAVAVVDLRLDLAEEVAHELVNKQIDAIAIQADVTKPDQVQTMVDAILSRWGTLTIGVNNAGIGLWADAESMAYTDWLRVIDIDLNSVFLCAQAEARVMLPAGYGKIINTASMSGHISNTPQNQAAYNTAKAGVIHLTRSLAAEWAKRGVRVNSISPGYTRTKLVDDLLATPIGQTMLPTWMGMTPMGRMAEVTDLQGAVVYLASSASDYMTGHDMVIDGGYCCW

SEQ ID NO: 3 - DNA, 1047 bp, Rasamsonia emersonii
  source 1..1047: mol_type = genomic DNA
atggctgccaacgtccccaagaccatgaaggctctcagatatgagaagcctgagacgttctccatcgtcgacattcccgttcccactctgcgtgagaacgatgtcttgatcaaggtcaaggcttgcggtgtctgtggtaccgacctgcacattcacgagggagaattccttgccaagttccctctcgttcctggccacgagactgtcggtgttgttgccgcagttggacccaaggtcaagggtttcgagatcggtgaccgtgttgttgccgacaactccgagctttgcggccaatgcttctactgccgacgaggagaggagttgctctgcgagcacttcgaagctcacggtgtcacgatgaacggcggtttcgctgagtactgcgcctaccctgccggccgtgtcttcaagatcaagaacctctctgacgtggacgccactctgcttgagcccgcgtcctgcgccgctcacggtctggacaagattgcccccaagatgggctcgtccgtcctggtgttcggcgccggtcccaccggtctggtccttgctcagatgctccgtctgaacggaggatgccgcgtcgtcgtcgctgcgcccgagggtctgaagatggacctggcccagaagctcggcgctggtgatgaatacgttgctctttctcgcacgaaccctcaggctcagtttgacaagctgaaggccgacaacccgtacggcttcgacattgtcgtcgaggctaccggcaatgccaagatcctggaggatgccatcaactatgtccgccgtggaggcaaactggtcgtgtacggtgtgtacgcgaacaaggaccgcgtctcgtggcccccgagcaagatcttcggtgacgaaatcaccattctgggtagcttctccgagacctacaagttccccgccgccatcgactacctggactccggcaaggtgaaggtccagggcatcgtgaacaagaccttccggctggagcagtgggaggagtgtctggcgtcgttgaagaacaagagcgccatcaaggcggcgatcgtctttgactaa

SEQ ID NO: 4 - protein, 348 aa, Rasamsonia emersonii
MAANVPKTMKALRYEKPETFSIVDIPVPTLRENDVLIKVKACGVCGTDLHIHEGEFLAKFPLVPGHETVGVVAAVGPKVKGFEIGDRVVADNSELCGQCFYCRRGEELLCEHFEAHGVTMNGGFAEYCAYPAGRVFKIKNLSDVDATLLEPASCAAHGLDKIAPKMGSSVLVFGAGPTGLVLAQMLRLNGGCRVVVAAPEGLKMDLAQKLGAGDEYVALSRTNPQAQFDKLKADNPYGFDIVVEATGNAKILEDAINYVRRGGKLVVYGVYANKDRVSWPPSKIFGDEITILGSFSETYKFPAAIDYLDSGKVKVQGIVNKTFRLEQWEECLASLKNKSAIKAAIVFD

SEQ ID NO: 5 - DNA, 786 bp, Bacillus spizizenii
  source 1..786: mol_type = genomic DNA
atgtatccagatttaaaaggaaaagttgtcgctattacaggagctgcttcaggattagggaaggcaatggccattcgcttcggcaaggagcaggcaaaagtggttatcaactactacagcaataagcaggatccgaacgaggtaaaggaagaggtcatcaaggcgggcggtgaagctgttgtcgtccaaggagacgtaacaaaagaggaagatgtaaaaaacatcgtccaaacagcgattaacgagttcggtacactcgatattatgattaataatgccggtcttgaaaatcccgttccttctcatgaaatgccgctgaaggattgggataaagtaatcagcacgaacttaacgggcgcctttttaggaagccgtgaagcgattaaatattttgttgaaaacgatataaaaggaaatgtcattaatatgtcgagcgtacatgaagtgattccgtggccattatttgttcactatgcggcaagtaaaggcggaatcaagctgatgacggaaacattggcgctggaatatgcgccgaaaggcattcgtgtcaacaatatcgggccaggcgcgatcaacacgccaatcaatgctgaaaaatttgctgatcctaagcagagagcagatgtagaaagcatgattccgatgggatatatcggtgaaccggaggaaattgcggcagtagcagcctggcttgcttcgaaggaagccagctacgtcacaggcatcacgttattcgcggacggcggtatgacccaatatccttccttccaggcaggacgcggataa

SEQ ID NO: 6 - protein, 261 aa, Bacillus spizizenii
MYPDLKGKVVAITGAASGLGKAMAIRFGKEQAKVVINYYSNKQDPNEVKEEVIKAGGEAVVVQGDVTKEEDVKNIVQTAINEFGTLDIMINNAGLENPVPSHEMPLKDWDKVISTNLTGAFLGSREAIKYFVENDIKGNVINMSSVHEVIPWPLFVHYAASKGGIKLMTETLALEYAPKGIRVNNIGPGAINTPINAEKFADPKQRADVESMIPMGYIGEPEEIAAVAAWLASKEASYVTGITLFADGGMTQYPSFQAGRG

SEQ ID NO: 7 - DNA, 858 bp, Spathaspora passalidarum
  source 1..858: mol_type = genomic DNA
atgactattcctaatactactgaaattacctcgtttacaaaccctgctcttggaccattacccacttcagcaccagaactagctactagtgtgcttgatttatttctgcttaagggcaaagtagcatcagttactggatcctctactggtatagggtacgctgtggcagaagcttttgcacaggcaggtgccgatgttgcaatttggtacaacaatcatcctgcaaccgaaaaggctgaaaaattggctaaaaaatatggcgttaaatgcagggcatacaaatgcaacatttccaatgctgacgatgttgatctgacgattaagcagattgagcaagattttggaacgattgatatctttgttgccaatgctggtgtggcttggacttctggttccatggttgatgtggacgataatcatgaaagttggcacaagattgtcgacttggatttgaatggagtatactattgttgtcattcaatagggaaagtattcaagaggaaaggaaagggttccttggttattacttcatcaatgtcgggcctgattgtcaatgttccacagctacaggctccttataatgctgctaaggctgctgtaaaacatttagctaaatcgttagcagttgaatgggctccatttgctagagtcaatagtatatctccaggatatatcactactgatatttctgaattcgccgatcctgaaatgaaggcaagatggtggcaattaactcctttggggagagagggattgccacaagaattagtaggagcttacttatacttagcctccaatgcctctacatttactacgggagccgatctcgttgtggacggtggatacactgctccataa

SEQ ID NO: 8 - protein, 285 aa, Spathaspora passalidarum
MTIPNTTEITSFTNPALGPLPTSAPELATSVLDLFLLKGKVASVTGSSTGIGYAVAEAFAQAGADVAIWYNNHPATEKAEKLAKKYGVKCRAYKCNISNADDVDLTIKQIEQDFGTIDIFVANAGVAWTSGSMVDVDDNHESWHKIVDLDLNGVYYCCHSIGKVFKRKGKGSLVITSSMSGLIVNVPQLQAPYNAAKAAVKHLAKSLAVEWAPFARVNSISPGYITTDISEFADPEMKARWWQLTPLGREGLPQELVGAYLYLASNASTFTTGADLVVDGGYTAP

SEQ ID NO: 9 - DNA, 768 bp, Gluconobacter oxidans
  source 1..768: mol_type = genomic DNA
atggaaaaactccgcctcgataaccgcgtcgccatcgtcaccggcggcgcacagaacatcggcctggcctgcgtcaccgcactggccgaggctggcgcccgcgtcatcatcgccgatctcgatgaagccatggccgcaaaagccgtggaagatctgcgcatggaaggccatgatgtcagcagcgtcgtcatggacgtcacgaacacggaaagtgtgcagaacgctgtccgcagcgttcacgagcaggaaggccgcgtggatatcctggtcgcctgcgccggcatctgcatttccgaagtcaaggccgaggacatgacggatggccagtggctcaagcaggtcgatatcaacctgaacggcatgttccgctcctgtcaggccgtcgggcgcatcatgctcgaacagaaacagggcgtcattgtcgcaatcggctccatgtccggcctgatcgtcaaccgcccgcagcagcaggcggcctataatgcgtcgaaagccggcgttcaccagtatatccgctcgctcgccgcagaatgggctccccatggcattcgtgccaacgccgttgcccccacctatatcgaaacgacgctcacccgcttcggcatggaaaagccggaactctacgacgcctggatcgccggaaccccgatgggccgcgtgggccagcccgatgaagtcgcttccgtcgtgcagttcctggcgtccgacgcagccagcctgatgaccggcgccatcgtgaatgtggatgcgggcttcaccgtctggtaa

SEQ ID NO: 10 - protein, 255 aa, Gluconobacter oxidans
MEKLRLDNRVAIVTGGAQNIGLACVTALAEAGARVIIADLDEAMAAKAVEDLRMEGHDVSSVVMDVTNTESVQNAVRSVHEQEGRVDILVACAGICISEVKAEDMTDGQWLKQVDINLNGMFRSCQAVGRIMLEQKQGVIVAIGSMSGLIVNRPQQQAAYNASKAGVHQYIRSLAAEWAPHGIRANAVAPTYIETTLTRFGMEKPELYDAWIAGTPMGRVGQPDEVASVVQFLASDAASLMTGAIVNVDAGFTVW

SEQ ID NO: 11 - DNA, 840 bp, Millerozyma farinosa
  source 1..840: mol_type = genomic DNA
atgtctcaagctatctcagcaatcaacgaaagagttggcccattgcccactaaagcaccccaactttcaaaaaacgtattagacctcttttccttgaaaggcaaggtagcatcggtgacaggttcatcgaagggaataggctttgctgtcgcagaagcatacgctcaagcaggggctgatgttgctttgtggtacaattcaagtcctgtcgaggataaggtaaaatacttgaaagagacctacggcgtgaaagtcaaagcctacaagtgtaacgtgggaaacagcgaggaggttgagaaaatagtagaccaaattgcagaggacttcggcacaattgacgtttttgttgctaatgcgggcattgcttggagcgaaggtagatctctcgaggttaagggttacgacgcttggcaaaaaattgtcgactacgacttgagcagtgtatactactgtgctaaggcagtgggtaagatcttccaagaaaagggctccggttcgttaatcctcaccgcatccatgtccggacacattgtgaatgttcctcaaatgcaggcaccatacaatgcggccaaggcggctgtgatccatctaggaaaatctttggctgtcgaatgggctccatttgccagggtcaacaccgtttctcctggctatattgccactgatattggtgaatttgtgctgcccgatgagaagaaaaagtggtggtcattgacacccttgggtagagaaggtcttcctcaagaactcactggtgcatacttgtattttgcttctaacgcatcgacctacactactggggctgacttggtcgtagacggtggctacactgtaccataa

SEQ ID NO: 12 - protein, 279 aa, Millerozyma farinosa
MSQAISAINERVGPLPTKAPQLSKNVLDLFSLKGKVASVTGSSKGIGFAVAEAYAQAGADVALWYNSSPVEDKVKYLKETYGVKVKAYKCNVGNSEEVEKIVDQIAEDFGTIDVFVANAGIAWSEGRSLEVKGYDAWQKIVDYDLSSVYYCAKAVGKIFQEKGSGSLILTASMSGHIVNVPQMQAPYNAAKAAVIHLGKSLAVEWAPFARVNTVSPGYIATDIGEFVLPDEKKKWWSLTPLGREGLPQELTGAYLYFASNASTYTTGADLVVDGGYTVP

SEQ ID NO: 13 - DNA, 840 bp, Debaryomyces fabryi
  source 1..840: mol_type = genomic DNA
atgtcacacccaacatcagttattaacgagcaggtaggcccattaccaactaaggccccacaactttctaagaatgtgaatgatttgttctcattaaagggtaaggtagcttctgtcaccggttcctcaggagggattggttgggctgtagctgaagcgtatgcccaagcaggagcagacgttgctgtttggtacaattctaaaaatgcagatgctaaggctgaatacttaactaagacttacggtgtgaagtcgaaggcatacaagtgtaacatttctgatccagaagacgtcgaaaaggtaattggacaaattgaaaaggatttcggtaccattgacgttttcgttgccaatgccggtgttccatggacagaaggtagaagtattgaagttgaaggatatgattcgtggaagaaggttatagatttggacttgagtggtgtctactattgcgctaaggctgtcggaaagattttcaagaagaatggtaagggttcgcttgtgttcacagcatcaatgtctggccacattgtgaatgttccacaattgcaagctccatacaacgccgcaaaggccggtgttttgcacttgagtaagtcattagctgtcgaatgggccccattcgctagagttaacaccatttcacctggttatattgccacagaaatttccgacttcgttccggacgatgtcaaggctaagtggtggcaattaattccattaggaagagaagctcttccacaagaattagttggtgcttacttatattttgcatccgatgcgtctacttacactacaggatcggacttattagtcgatggtggttactctgctccataa

SEQ ID NO: 14 - protein, 279 aa, Debaryomyces fabryi
MSHPTSVINEQVGPLPTKAPQLSKNVNDLFSLKGKVASVTGSSGGIGWAVAEAYAQAGADVAVWYNSKNADAKAEYLTKTYGVKSKAYKCNISDPEDVEKVIGQIEKDFGTIDVFVANAGVPWTEGRSIEVEGYDSWKKVIDLDLSGVYYCAKAVGKIFKKNGKGSLVFTASMSGHIVNVPQLQAPYNAAKAGVLHLSKSLAVEWAPFARVNTISPGYIATEISDFVPDDVKAKWWQLIPLGREALPQELVGAYLYFASDASTYTTGSDLLVDGGYSAP

SEQ ID NO: 15 - DNA, 849 bp, Torulaspora delbrueckii
  source 1..849: mol_type = genomic DNA
atgggcaagactgtagcaactggtattgaaaccccccaaccttatccacaattgcctgaacatgttatggacatgttttcattaaaaggtaaggtggcctctgtgactggtgcttccggtggtattggttatgaagtggccgttgcatttgcacaggcaggtgccaatgttgctatgtggtacaactcgcattctgtcgaggaggaagctgaaaagctgtcgaagaaatacaacgtcaccgttaaggcttataaatgttcattgactgatactaaggctgttgaagaaactgtgcaacagattaagaaggactttggtggtagaattgatataatggtggctaatgctggtgtggcgtgggataagggtcccttgaccgagctggccgaaaaagattctgagctttgtgacaaggaatggcagaaagttttgagcatcgatatcaatggtgtctataatgcagccaagagtattggtccaattttcaagaagcaaggatcaggttctttcattgcaactggttcgatgtctggtcacattgcaaacgttccgcaattacaggtcgcatacaatactgcaaaggctgccgttattcatatgtgcaaatcgctggctgtcgaatggacaggttacgcccgtgctaacactgtatccccaggttacgtagccactccattgaatgctgggatggacgaagagatgctaaagaaatggaacaccttagtcccactaggtcgtctcgctttgcccaaggaaatggttggtgcttatttgtacttggcctctaatgcctcaacatacacgaccggtagcgacatactagtggatggtggttactgtagtgtttaa

SEQ ID NO: 16 - protein, 282 aa, Torulaspora delbrueckii
MGKTVATGIETPQPYPQLPEHVMDMFSLKGKVASVTGASGGIGYEVAVAFAQAGANVAMWYNSHSVEEEAEKLSKKYNVTVKAYKCSLTDTKAVEETVQQIKKDFGGRIDIMVANAGVAWDKGPLTELAEKDSELCDKEWQKVLSIDINGVYNAAKSIGPIFKKQGSGSFIATGSMSGHIANVPQLQVAYNTAKAAVIHMCKSLAVEWTGYARANTVSPGYVATPLNAGMDEEMLKKWNTLVPLGRLALPKEMVGAYLYLASNASTYTTGSDILVDGGYCSV

SEQ ID NO: 17 - DNA, 789 bp, Sphaerochaeta pleomorpha
  source 1..789: mol_type = genomic DNA
atggttgataagattctggataggttcagtttaaaagggaaaaccgcattggtgacaggtggtggtcagggtattggacgcggatatgcttttgcgctgggagaagccggggccaaggtagccattgttgatatcaatggagaaactgctgaagcgacagccaaggacctgcaagcagcggggattgcatcaatgtcctatgtctgtgatgtaactaatcccgatcaggtgatggcaatggtgaaacatattgtcaaagaatgggggactttgactataggggttaacaacgctggcatgggaatctggagcgatgcagtaaccatgccctatgagatgtggagaaagaccatggctttaaacatggatgggatttttctttgtgccagggaggaggcaaagtatatgatacgcgaggggtatgggaaaattatcaatactgcttcgatgagcgcccatatatcgaataccccacagaatcaagtagcttacaatgcatccaaggcgggggttctccacatgaccagatctttggccgcagaatgggctccctatggcgtgagggtgaactccatcagtcccggatatacaaggacagagctggtcgaaaagctgcttgcaactccagaggggaaaaaaatggaatcagactggttgcaactgattccccaaaaaaagatggctaccgtcgaggatttgcaaggcgcttgcttgtaccttgcctgcagggcttcggattacacaacaggtagcgatattctcatagatggtgggtattgctgttggtaa

SEQ ID NO: 18 - protein, 262 aa, Sphaerochaeta pleomorpha
MVDKILDRFSLKGKTALVTGGGQGIGRGYAFALGEAGAKVAIVDINGETAEATAKDLQAAGIASMSYVCDVTNPDQVMAMVKHIVKEWGTLTIGVNNAGMGIWSDAVTMPYEMWRKTMALNMDGIFLCAREEAKYMIREGYGKIINTASMSAHISNTPQNQVAYNASKAGVLHMTRSLAAEWAPYGVRVNSISPGYTRTELVEKLLATPEGKKMESDWLQLIPQKKMATVEDLQGACLYLACRASDYTTGSDILIDGGYCCW

SEQ ID NO: 19 - DNA, 789 bp, Leadbettera azotonutricia
  source 1..789: mol_type = genomic DNA
atggcagaaaaaattcttgacagattcaagctggacggcaagaccgccctggttaccggaggagggcagggcattggccaggcctactgtttcgccctgggcgaagcaggcgcaaagatagctgtggtagacatcaacaccaccgttgcggaagagaccgcccaggccctgacaaaaaaaggcatagaggctattgcgataaccacagatgttacaaaagaagacgaagtaataaagatggttaaaaccgtcatagacaaatggggcttcctcaccataggcgtcaacaacgccggcatgggtgtctggcgcgatgcgttaacccaggactttgccgaatggcggaagatcctctccctcaacctcgattcgatcttcctctgctctcgcaccgaagccgtggaaatggcaaagaaaggctatggcaagatcgtcaacaccgcttccatgtccgcccatatttccaacaccccccagaaccaggcagcctacaacagctccaaagccggtgtgctccatctgacccgcagcctcgctgccgaatgggcgcccaaaaatatcagggtaaacagcatctccccgggttatacaaaaaccgccctggtagacaagctcctcgaaaccccggaaggcaaaaccatgctgcccaagtggctggaaaaagtccccatggggcgcatggctaccgtggaagatctgcaaggcgccgttgtgtatctggcatcgcccgcatcggattatgccaccggcacagatattatcattgacggcggatattgctgctggtaa

SEQ ID NO: 20 - protein, 262 aa, Leadbettera azotonutricia
MAEKILDRFKLDGKTALVTGGGQGIGQAYCFALGEAGAKIAVVDINTTVAEETAQALTKKGIEAIAITTDVTKEDEVIKMVKTVIDKWGFLTIGVNNAGMGVWRDALTQDFAEWRKILSLNLDSIFLCSRTEAVEMAKKGYGKIVNTASMSAHISNTPQNQAAYNSSKAGVLHLTRSLAAEWAPKNIRVNSISPGYTKTALVDKLLETPEGKTMLPKWLEKVPMGRMATVEDLQGAVVYLASPASDYATGTDIIIDGGYCCW

SEQ ID NO: 21 - DNA, 792 bp, Rasamsonia emersonii
  source 1..792: mol_type = genomic DNA
atggttctctctcagcccgaaaacaagcatgttatgaaagccttcgatttgaccggcaaggtcgcagctgtgacaggaggagctcgcgggatcggtcttgaggtctccagagggcttgcagaagctggtgcgaatgttgcagtcatctacagctcgtccaagaatgccgacgccgttgcagccgagatcgcagctgccaacaacgtcaagacagccgcatacaaggcagatgtaagcaaccagcaggatatcgagagcaccattcagcagatagcaaaggactttggaaaactggacataatcgtggcgaattctggaatcgcaagcacgcatcccgctgaagactacacagtggaggagtggagagatatcctcaaagtgaatctagatggagcgttctacacagcgcaggcggcggcaaggatcttcaagacgcaagggcatggaaacgtcatcttcacggcttctgtcagcgcaaggctggtcaatgtcccccagaaacaagctgcgtacaacgcctcgaaagctggtcttgtccagctggcaaagtgcttgtcagtggaatggatagactattgtcgtgtcaactgcatttcgcctggttttattgcgacagaaatcttggatatccaccccaaggagtggagagaaaaatggctcgacatggttcccgcgcgtcggatggctgctacttacgagctaaagggggcgtatgttttctgtgcttctgacgcgtcaagctacatgaccggcgctgatatagtcattgatggtggatatacgcttccgtaa

SEQ ID NO: 22 - protein, 263 aa, Rasamsonia emersonii
MVLSQPENKHVMKAFDLTGKVAAVTGGARGIGLEVSRGLAEAGANVAVIYSSSKNADAVAAEIAAANNVKTAAYKADVSNQQDIESTIQQIAKDFGKLDIIVANSGIASTHPAEDYTVEEWRDILKVNLDGAFYTAQAAARIFKTQGHGNVIFTASVSARLVNVPQKQAAYNASKAGLVQLAKCLSVEWIDYCRVNCISPGFIATEILDIHPKEWREKWLDMVPARRMAATYELKGAYVFCASDASSYMTGADIVIDGGYTLP

SEQ ID NO: 23 - DNA, 882 bp, Rasamsonia emersonii
  source 1..882: mol_type = genomic DNA
atggctgcgtctcccatcaccaacggcctcttcaaccatgacaactccaccccgcccgagcatcccagcctgttcgcgctgttctcgctcaagggcaagaccgccatcgtcacgggcgccggtgctggcatcggcctgcatgtggctcatggtctggctgaggctggcgccaacgttgcgctcttctacaacaccaacaccaagacgccggagcgcgctgccgagatcgagcagcagtatggcgtgaaggccaaggcataccaggtcgacgtccgcgacgccaagaagctcgaggagacggtcaaccaggccgtgcgcgacctcaacggccgcctggacatcttcatcgccaacgccggcatcccgtggaccaagggccccagcgttgacggcccgctggaccactaccaggccgtcgtgcagaccgacctggacggcgtcttctactcggccaaggcggcggcagcccactggcggcgccagaaggaggagggcaccgacctgttcggcaacaagctgcagaacttcacctacggcagcttcgtggccacggcgtcgatgagcggccatatcgtgaacattccccagctgcaggcggcgtacaacgccgccaaggcgggcgtcatccacatggtcaaatcgtttgccgtcgaatgggcgcgcttcgcccgtgccaactcggtctctcctggctacatcgccacggagatctccaacttcgttcccgcggagaccaagaagctctggcgggacaagacgccgctgggccgcgagggtctgccgcaggagctcaagggcgcctacctgtacctggccagcgatgcggccagcttcacgacgggcgcggacctggtggtcgacggaggctacaccctgccttaa

SEQ ID NO: 24 - protein, 293 aa, Rasamsonia emersonii
MAASPITNGLFNHDNSTPPEHPSLFALFSLKGKTAIVTGAGAGIGLHVAHGLAEAGANVALFYNTNTKTPERAAEIEQQYGVKAKAYQVDVRDAKKLEETVNQAVRDLNGRLDIFIANAGIPWTKGPSVDGPLDHYQAVVQTDLDGVFYSAKAAAAHWRRQKEEGTDLFGNKLQNFTYGSFVATASMSGHIVNIPQLQAAYNAAKAGVIHMVKSFAVEWARFARANSVSPGYIATEISNFVPAETKKLWRDKTPLGREGLPQELKGAYLYLASDAASFTTGADLVVDGGYTLP

SEQ ID NO: 25 - DNA, 1206 bp, Starkeya novella
  source 1..1206: mol_type = genomic DNA
atggcgaaaatactttgcgttctctatgacgatccggtcgacggctacccgaagacctatgcgcgcgacgacctgccgaagatcgaccactatccgggcgggcagacgctgcccacgcccaaggcgatcgacttcacgccgggcgcgctgctcggctcggtctccggcgagctcggcctgcgcaaatacctggaagccaacggccataccttcgtcgtcacctccgataaggacggcccggattcggtgttcgagagggaactcgtcgacgccgacgtggtgatctcgcagcccttctggccggcctatctgacgcccgagcgcatcgccaaggcgaagaacctgaagctcgcgctcaccgccggcatcggctccgatcatgtcgatcttcagtcagctatcgaccgtggcatcactgtggccgaagtcacatattgcaactcgatcagcgtcgccgagcacgtggtgatgatgatcctcggcctggtacgaaactacattccctcgcatgactgggcgcgcaagggcggctggaacatagccgactgcgtagagcactcctacgacctcgagggcatgaccgtcggctcggtggccgccggccgcatcggcctcgccgtgctgcgccgcctcgcgccgttcgacgtgaagctgcactataccgaccgccaccgtctgccagaagcggtcgagaaggagctgggcctcgtctggcacgatacccgcgaggacatgtacccgcattgcgacgtggtcacgctcaacgtgccgctgcaccccgaaaccgagcacatgatcaatgacgagacgctgaagctgttcaagcgcggcgcctatatcgtcaacaccgcccgcggcaagctcgccgaccgcgacgccatcgtccgcgcgatcgagagcgggcagctcgcgggctatgccggcgacgtgtggttcccgcagccggctccgaaggaccacccctggcgcaccatgaagtgggaaggcatgacgccgcacatctccggcacctcgctctctgcccaggcgcgctacgcggcgggcacgcgcgagatcctcgaatgcttcttcgagggccggccgatccgcgacgagtacctgatcgtgcagggcggcgcgctcgccggcaccggcgcgcattcctactcgaagggcaatgcgaccggcggttcggaagaggccgcgaagttcaagaaggctggctga

SEQ ID NO: 26 - protein, 401 aa, Starkeya novella
MAKILCVLYDDPVDGYPKTYARDDLPKIDHYPGGQTLPTPKAIDFTPGALLGSVSGELGLRKYLEANGHTFVVTSDKDGPDSVFERELVDADVVISQPFWPAYLTPERIAKAKNLKLALTAGIGSDHVDLQSAIDRGITVAEVTYCNSISVAEHVVMMILGLVRNYIPSHDWARKGGWNIADCVEHSYDLEGMTVGSVAAGRIGLAVLRRLAPFDVKLHYTDRHRLPEAVEKELGLVWHDTREDMYPHCDVVTLNVPLHPETEHMINDETLKLFKRGAYIVNTARGKLADRDAIVRAIESGQLAGYAGDVWFPQPAPKDHPWRTMKWEGMTPHISGTSLSAQARYAAGTREILECFFEGRPIRDEYLIVQGGALAGTGAHSYSKGNATGGSEEAAKFKKAG

SEQ ID NO: 27 - DNA, 1356 bp, Carnobacterium divergens
  source 1..1356: mol_type = genomic DNA
atgaaagtagtagtagtaggctgtacacatgcaggaacagcggcagttaagacgattttaaatgaacatccagatgcatcagtatcagtatatgagcgtaatgacaatgtctcatttctatcttgtgggattgcgttgtatgttggtggagttgtgaaagatcctgcaggtttgttttattcaagtccagaagaacttgcatcaatgggcgcgaaaattaacatggaacacaatgtgaaaaatatagataatgagaataaggtcgtagtaattgagaatttaaaaacaggcgaaacatttgaagaaagctatgataagttggtaatgacaactggatcatggccaattattcctccaattgatggaatcaatagtgaaaatattcttttgtgtaaaaactataaccaagcaaatgaaattattaaagaatcaaaaaatgctaaaaagattgtcattgttggtggtggctatattggaattgaattagttgaggcatttgcagaatctggcaagcaagtgacgctagttgatggattagatcgtattttaaacaaatatttagatgctgaattcacttctgttttagagcatgatttacaagaaagaggcgttacgctagctttaaaccaaaccgtcgagaaatttgttgccaatgaatcaggtgctgtgacagctgtgaaaacaccagttggagaatatgaggctgatttagttattttatgtgttggatttaaaccaaatactgatttgttgaaggataaagtagagatgttgccaaatggtgccatcgtagtggatgaatatatgagaacaagcgatgaagcgatttttgctgctggcgatagttgcgcggttcattataatccaactggaggctctgcgtatattccgttagctacaaatgcagttagaatgggagctttagttgggaaaaatattgtttctccaacagttaaatatcgtggcacgcaagcaacttctggtttatatttatttggttttaatataggttcaaccggattgactgaaaatagcgctcctcattttggcgtagaggttcgttcagtagttgtagaagataattatcgtccagagtttatgccgacaacagagaaagtaacgatgaaattagtttatgaagtaggaacgaatcggattgttggaggtcaaatcatgtcaaaatatgatgtgacacaatctgccaatacgttatctttatgtgttcaaaataaaatgacgattgaggatttggcttatgtagatttcttcttccaacctcactttgatcgtccttggaactatttaaatattttagcgcaagcagctgttgagcaagagcgtaaactagcaaaataa

SEQ ID NO: 28 - protein, 451 aa, Carnobacterium divergens
MKVVVVGCTHAGTAAVKTILNEHPDASVSVYERNDNVSFLSCGIALYVGGVVKDPAGLFYSSPEELASMGAKINMEHNVKNIDNENKVVVIENLKTGETFEESYDKLVMTTGSWPIIPPIDGINSENILLCKNYNQANEIIKESKNAKKIVIVGGGYIGIELVEAFAESGKQVTLVDGLDRILNKYLDAEFTSVLEHDLQERGVTLALNQTVEKFVANESGAVTAVKTPVGEYEADLVILCVGFKPNTDLLKDKVEMLPNGAIVVDEYMRTSDEAIFAAGDSCAVHYNPTGGSAYIPLATNAVRMGALVGKNIVSPTVKYRGTQATSGLYLFGFNIGSTGLTENSAPHFGVEVRSVVVEDNYRPEFMPTTEKVTMKLVYEVGTNRIVGGQIMSKYDVTQSANTLSLCVQNKMTIEDLAYVDFFFQPHFDRPWNYLNILAQAAVEQERKLAK

SEQ ID NO: 29 - DNA, 1374 bp, Streptococcus mutans
  source 1..1374: mol_type = genomic DNA
atgagcaaaattgttatcgtgggtgcaaatcatgcaggcaccgcagcaattaataccattctggataattatggcagcgaaaatgaagtggttgtgtttgatcagaatagcaacattagctttctgggttgtggtatggcactgtggattggtaaacaaattagcggtccgcagggtctgttttatgcagataaagaaagcctggaagcaaaaggtgccaaaatctatatggaaagtccggttaccgccattgattatgatgcaaaacgtgttaccgcactggttaatggtcaagaacatgttgaaagctacgagaaactgattctggcaaccggtagcaccccgattctgcctccgattaaaggtgcagccattaaagaaggtagtcgcgattttgaagcaaccctgaaaaatctgcagttcgtgaaactgtatcagaatgccgaagatgtgattaacaaactgcaggataaaagccagaatctgaatcgtattgcagttgttggtgcaggttatattggtgttgaactggcagaagcatttaaacgtctgggtaaagaagtgattctgattgacgttgttgatacctgtctggcaggttattatgatcaggatctgagcgaaatgatgcgtcagaatctggaagatcatggtatcgaactggcatttggtgaaaccgttaaagcaattgaaggtgatggtaaagtggaacgtattgttaccgataaagcaagccatgatgtggatatggttattctggcagttggttttcgtccgaatacagcactgggtaatgcaaaactgaaaacctttcgtaatggtgcctttctggtggataaaaaacaagaaaccagcatcccggatgtttatgcaattggtgattgtgcaaccgtgtatgataatgccattaacgacaccaactatattgcactggcaagcaatgcactgcgtagcggtattgttgcaggtcataatgcagccggtcataaactggaaagtctgggtgttcagggtagcaatggtatttcaatttttggcctgaatatggttagcaccggtctgacccaagaaaaagccaaacgttttggttataatccggaagttaccgcctttaccgattttcagaaagccagctttatcgagcatgataactatccggttacgctgaaaattgtgtatgacaaagatagccgtctggttctgggtgcacagatggccagcaaagaagatatgagcatgggtattcacatgtttagcctggccattcaagagaaagttaccattgaacgtctggccctgctggattatttctttctgccgcattttaatcagccgtacaactatatgaccaaagcagcactgaaagccaaataa

SEQ ID NO: 30 - protein, 457 aa, Streptococcus mutans
MSKIVIVGANHAGTAAINTILDNYGSENEVVVFDQNSNISFLGCGMALWIGKQISGPQGLFYADKESLEAKGAKIYMESPVTAIDYDAKRVTALVNGQEHVESYEKLILATGSTPILPPIKGAAIKEGSRDFEATLKNLQFVKLYQNAEDVINKLQDKSQNLNRIAVVGAGYIGVELAEAFKRLGKEVILIDVVDTCLAGYYDQDLSEMMRQNLEDHGIELAFGETVKAIEGDGKVERIVTDKASHDVDMVILAVGFRPNTALGNAKLKTFRNGAFLVDKKQETSIPDVYAIGDCATVYDNAINDTNYIALASNALRSGIVAGHNAAGHKLESLGVQGSNGISIFGLNMVSTGLTQEKAKRFGYNPEVTAFTDFQKASFIEHDNYPVTLKIVYDKDSRLVLGAQMASKEDMSMGIHMFSLAIQEKVTIERLALLDYFFLPHFNQPYNYMTKAALKAK

SEQ ID NO: 31 - DNA, 1356 bp, synthetic construct
  source 1..1356: mol_type = other DNA
atgaaagtagtagtagtaggctgtacacatgcaggaacagcggcagttaagacgattttaaatgaacatccagatgcatcagtatcagtatatgagcgtaatgacaatgtctcatttctatcttgtgggattgcgttgtatgttggtggagttgtgaaagatcctgcaggtttgttttattcaagtccagaagaacttgcatcaatgggcgcgaaaattaacatggaacacaatgtgaaaaatatagataatgagaataaggtcgtagtaattgagaatttaaaaacaggcgaaacatttgaagaaagctatgataagttggtaatgacaactggatcatggccaattattcctccaattgatggaatcaatagtgaaaatattcttttgtgtaaaaactataaccaagcaaatgaaattattaaagaatcaaaaaatgctaaaaagattgtcattgttggtggtggctatattgcgattgaattagttgaggcatttgcagaatctggcaagcaagtgacgctagttgcgcgtagcgatcgtattttacgtaaatatttagatgctgaattcacttctgttttagagcatgatttacaagaaagaggcgttacgctagctttaaaccaaaccgtcgagaaatttgttgccaatgaatcaggtgctgtgacagctgtgaaaacaccagttggagaatatgaggctgatttagttattttatgtgttggatttaaaccaaatactgatttgttgaaggataaagtagagatgttgccaaatggtgccatcgtagtggatgaatatatgagaacaagcgatgaagcgatttttgctgctggcgatagttgcgcggttcattataatccaactggaggctctgcgtatattccgttagctacaaatgcagttagaatgggagctttagttgggaaaaatattgtttctccaacagttaaatatcgtggcacgcaagcaacttctggtttatatttatttggttttaatataggttcaaccggattgactgaaaatagcgctcctcattttggcgtagaggttcgttcagtagttgtagaagataattatcgtccagagtttatgccgacaacagagaaagtaacgatgaaattagtttatgaagtaggaacgaatcggattgttggaggtcaaatcatgtcaaaatatgatgtgacacaatctgccaatacgttatctttatgtgttcaaaataaaatgacgattgaggatttggcttatgtagatttcttcttccaacctcactttgatcgtccttggaactatttaaatattttagcgcaagcagctgttgagcaagagcgtaaactagcaaaataa

SEQ ID NO: 32 - protein, 451 aa, synthetic construct
MKVVVVGCTHAGTAAVKTILNEHPDASVSVYERNDNVSFLSCGIALYVGGVVKDPAGLFYSSPEELASMGAKINMEHNVKNIDNENKVVVIENLKTGETFEESYDKLVMTTGSWPIIPPIDGINSENILLCKNYNQANEIIKESKNAKKIVIVGGGYIAIELVEAFAESGKQVTLVARSDRILRKYLDAEFTSVLEHDLQERGVTLALNQTVEKFVANESGAVTAVKTPVGEYEADLVILCVGFKPNTDLLKDKVEMLPNGAIVVDEYMRTSDEAIFAAGDSCAVHYNPTGGSAYIPLATNAVRMGALVGKNIVSPTVKYRGTQATSGLYLFGFNIGSTGLTENSAPHFGVEVRSVVVEDNYRPEFMPTTEKVTMKLVYEVGTNRIVGGQIMSKYDVTQSANTLSLCVQNKMTIEDLAYVDFFFQPHFDRPWNYLNILAQAAVEQERKLAK

(19) United States
(12) Patent Application Publication
(10) Pub. No.: US 2026/0308649 A1
(43) Pub. Date: Oct. 8, 2026

(54) METHOD FOR PRODUCING D-TALITOL, D-TAGATOSE, AND D-PSICOSE

(71) Applicant: ANNIKKI GMBH, Raaba-Grambach (AT)

(72) Inventors: Nicole STAUNIG, Raaba-Grambach (AT); Maria DUPONT, Raaba-Grambach (AT); Melanie TROBE, Raaba-Grambach (AT)

(21) Appl. No.: 19/481,641

(22) PCT Filed: May 15, 2024
(86) PCT No.: PCT/EP2024/063389

§ 371 Date: Nov. 5, 2025

(30) Foreign Application Priority Data
May 15, 2023 (EP) 23173450.0
Oct. 27, 2023 (EP) 23206404.8

(51) Int. Cl. C12P 19/02; C12N 9/02; C12N 9/04; C12N 9/90; C12P 19/24

(52) U.S. Cl. CPC C12P 19/02; C12N 9/0006; C12N 9/0036; C12N 9/0093; C12N 9/90; C12P 19/24; C12Y 101/01001; C12Y 101/01047; C12Y 106/03001; C12Y 117/01; C12Y 501/03

(57) ABSTRACT

The invention consists of a method for the production of an aqueous solution containing D-talitol in that D-psicose is formed in vitro from D-fructose, which exists in a dissolved state in an aqueous solution, by treating it with an epimerase, whereupon the D-psicose is reduced in vitro to D-talitol by treating it with an NAD(P)H-dependent oxidoreductase. The D-talitol contained in the aqueous solution can easily be oxidized to D-tagatose or D-psicose by treating it with an oxidoreductase.

The present invention relates to the preparation of D-talitol. Furthermore, the invention relates to the preparation of an aqueous solution containing D-tagatose or D-psicose.

### BACKGROUND OF THE INVENTION

The hexavalent sugar alcohol D-talitol (also known as D-altritol) belongs to the so-called rare sugars due to its low natural occurrence (e.g., in brown algae such as Himanthalia elongata (Reed et al., 1995)). Because of the lack of a simple production method for the sugar alcohol, the possible applications of D-talitol have not been well studied (Li et al., 2013).

D-Talitol plays an important role in the so-called Izumoring strategy, as its oxidation leads to the ketohexoses D-tagatose and D-psicose, as well as to the aldohexoses D-altrose and D-talose, which are likewise counted among the rare sugars (Izumori, 2006).

D-Tagatose is found in the rubber of the tropical tree Sterculia setigera (Hirst et al., 1949), in the lichen Roccella (Lindberg, 1955), and in highly heated milk (Adachi, 1958), among other things. D-Psicose has been detected in the leaves of rosemary-leaved willows (Itea sp.) (Hough & Stacey, 1963), but is also found in processed foods such as confectionery and spicy sauces, where it is formed under the influence of heat from D-fructose, its C3 epimer, (Oshima et al., 2006).

Both D-tagatose and D-psicose are characterized by their sweet taste with an energy content that is significantly lower (relative to sucrose) (Jiang et al., 2020; Oh, 2007; Roy et al., 2018), which makes both sugars particularly interesting as sweeteners for the food industry.

D-tagatose and D-psicose have been approved by the U.S. Food and Drug Administration (FDA) as Generally Recognized as Safe (GRAS) sweeteners. However, in the European Union, only D-tagatose is on the so-called Novel Foods List and is therefore permitted for use as a sweetener in the EU (Levin, 2002; Ahmed et al., 2022).

D-tagatose can also be used as a fungicide—it inhibits the D-mannose metabolism of fungi and oomycetes and is thus effective against downy mildew (Mochizuki et al., 2020). U.S. Pat. No. 10,638,752 B2 and Chahed et al. (2021) describe the use of D-tagatose against various pathogens of plant diseases.

D-psicose has a positive (e.g., antidiabetic) influence on lipid metabolism and carbohydrate metabolism and has anti-inflammatory and antioxidant effects (Zhang et al., 2016; Jiang et al., 2020; Chen et al., 2022).

There are several chemical or biotechnological routes for the production of D-tagatose, such as, for example, the base-induced isomerization of D-galactose obtained from lactose (EP 0518874 B1; U.S. Pat. Nos. 9,150,938 B2; 8,802,843 B2), the enzymatic isomerization of D-galactose using L-arabinose isomerase (Heath et al., 1958; Oh, 2007; Roy et al., 2018), production beginning with mono-, di-, oligo- or polysaccharides via phosphorylated intermediates with the C4-epimerization of D-fructose 6-phosphate to D-tagatose 6-phosphate as the main step (EP 3322803 B1; U.S. Pat. Nos. 10,138,506 B2; 11,034,988 B2; Dai et al., 2020; Lee et al., 2017) or the oxidation of the sugar alcohol galactitol with galactitol 2-dehydrogenase (EC 1.1.1.16) (Roy et al., 2018; Shaw, 1956).

Via C4 epimerization, D-tagatose can be produced directly from D-fructose. By specifically optimizing the enzyme tagaturonate 3-epimerase from Thermotoga petrophila, a tagatose 4-epimerase (T4E) could be developed which produced 213 g/l D-tagatose from 700 g/l D-fructose at 80° C. in the presence of 1.5 mM Ni²⁺ within 2 hours (Shin et al., 2020). Other epimerases derived from thermophilic microorganisms are also known (e.g., EP 3006568 B1; EP 3211078 B1; U.S. Pat. Nos. 10,196,626 B2; 11,180,785 B2; 11,408,017 B2; EP 3677675 A1), but they also require elevated reaction temperatures (>50° C.) and the addition of Zn−, Ni− or Mn− ions for optimum activity.

In a recent study, tagatose 4-epimerases from Thermotoga neapolitana and from Kosmotoga olearia were tested. For this purpose, they were expressed in Corynebacterium glutamicum and were used in the form of whole-cell biocatalysts. In this way, conversions of 13.8% D-tagatose (T4E from T. neapolitana) and, respectively, of 14.4% D-tagatose (T4E from K. olearia; 21.7% after codon optimization) could be achieved (30 g/l D-fructose as a substrate, 60° C. for 2.5 h), however, heavy metal salts (1 mM NiSO₄ for T. neapolitana and 3 mM CoSO₄ for K. olearia) must also be added (Jeon et al., 2023).

Since D-tagatose is a sweetener, heavy metal ions are undesirable in the finished product. The higher reaction temperatures and incomplete conversion are further disadvantages of C4 epimerization.

On the other hand, D-psicose can be obtained by epimerizing D-fructose with a ketose 3-epimerase (Izumori et al., 1993). Ketose 3-epimerases can be divided into three groups depending on their substrate specificity: 1) D-tagatose 3-epimerase (DTE), 2) D-psicose 3-epimerase (DPE) or D-allulose 3-epimerase (DAE), and 3) L-ribulose 3-epimerase (LRE) (Zhang et al., 2016; Jiang et al., 2020).

However, the conversion of D-fructose to D-psicose by means of ketose 3-epimerases does not proceed completely; rather, an equilibrium ratio is formed between the two epimers. Depending on the reaction conditions (temperature between 4° and 70° C., pH between 6 and 11), this ratio ranges between 80:20 and 62.5:37.5 (D-fructose:D-psicose). In addition, many of the epimerases require a divalent metal ion such as Mn²⁺ or Co²⁺ (toxic) as a cofactor (Zhang et al., 2016; Jiang et al., 2020).

The D-fructose/D-psicose mixtures emerging during the epimerization of D-fructose must be separated either by chromatographic processes or by the “biological method” (fermentation of excess D-fructose to ethanol, for example) (Jiang et al., 2020). In US 2021/0189441 A1, excess D-fructose is converted to L-lactic acid by a probiotic microorganism (Lactobacillus or Saccharomyces). EP 3423460 B1 describes a process involving ion exchange and continuous chromatography for the purification of a D-fructose/D-psicose mixture and for obtaining high-purity D-psicose. EP 3553069 A1 and Van Duc Long et al. (2009) disclose a method for the separation of D-psicose and D-fructose based on simulated moving bed chromatography.

There are several other biotechnological routes for the production of D-psicose, such as, for example, production beginning with mono-, di-, oligo- or polysaccharides via phosphorylated intermediates with the C3 epimerization of D-fructose 6-phosphate to D-psicose 6-phosphate as the main step (Li et al., 2021; U.S. Pat. No. 11,168,342 B2; 10,907,182 B2) or the aldol reaction of dihydroxyacetone phosphate and D-glyceraldehyde with subsequent dephosphorylation of D-psicose 1-phosphate (Wang et al., 2020; Li et al., 2020).

The unfavourable state of the equilibrium of the epimerization of D-fructose to D-psicose can also be positively influenced by downstream reactions. One possibility is the specific phosphorylation of D-psicose to D-psicose 1-phosphate with L-rhamnulose kinase and subsequent dephosphorylation with an acidic phosphatase (Xiao et al., 2019). The drawback of this process is the stoichiometric consumption of the expensive phosphate donor adenosine triphosphate (ATP).

An alternative thereto is the combination of epimerase with a ribitol dehydrogenase (RDH) and formate dehydrogenase (FDH; oxidizes formate to CO₂) as a regenerative enzyme for the cofactor nicotinamide adenine dinucleotide (NADH), by means of which D-fructose is converted to allitol via D-psicose (Takeshita et al., 2000). Through a subsequent oxidation step, allitol can be converted back to D-psicose.

Wang et al. (2023) describe a system consisting of two E. coli whole-cell biocatalysts for the conversion of D-fructose (90 g/l) to D-psicose with a turnover of 90%. In the first step (conversion of D-fructose to allitol), E. coli cells containing a DPE from Clostridiales, an RDH from Providencia alcalifaciens, an FDH from Starkeya and another DPE from Rhizobium straminoryzae were used. In this way, D-fructose (500 mM=90 g/l) was converted to 452 mM allitol (turnover of 90.4%) within 12 hours at 37° C. and pH 6 using 1000 mM sodium formate (two equivalents based on D-fructose). Approximately 30 mM D-sorbitol (the reduction product of D-fructose) emerged as a byproduct. The cells were separated by centrifugation, and leaked proteins in the allitol-containing supernatant were deactivated through heat exposure. E. coli cells containing an RDH from Rubrivivax sp. and an NADH oxidase from Streptococcus pyogenes were then added to the allitol solution. Allitol (452 mM) was oxidized to D-psicose (450 mM) at pH 7 within 24 hours, and the byproduct D-sorbitol was oxidized back to D-fructose. The total turnover of the process was thus 90%.

In an article, Izumori (2002) describes a toolbox composed of an epimerase (D-tagatose 3-epimerase) and oxidoreductases for the bioproduction of ketohexoses with hexitols as intermediates (known as the Izumoring strategy). The examples cited in the article for the production of hexitols from ketohexoses and vice versa use exclusively microbial cells as catalysts (Izumori, 2002).

By heterogeneous catalytic hydrogenation of D-psicose on Raney nickel or metals from the platinum group, a mixture of the epimers allitol and D-talitol is obtained, which must be separated chromatographically (US 2004/0143024 A1). In contrast, the use of microorganisms allows the stereoselective reduction of ketoses to sugar alcohols (preferential formation of one of the two epimers).

In general, it can be said that in recent years there has been a trend towards whole-cell processes (microbial and fermentative processes) in bioproduction processes (cf. Wang et al., 2023). For example, according to Boverio et al. (2023), isolated (uronate) dehydrogenases (“in vitro”) have “inherent disadvantages,” and industrial use is regarded as “impractical” by Vastano et al. (2019) due to the demand for cofactor (NADH).

For example, D-talitol can be produced by reducing D-tagatose with the fungus Aureobasidium pullulans (strain 113B) (Muniruzzaman et al., 1994). The reverse reaction can be accomplished using altritol 5-dehydrogenase (EC 1.1.1.407), which is part of the D-altritol catabolism in Agrobacterium tumefaciens C58 (Wichelecki et al., 2015).

For example, D-talitol can be oxidized to D-psicose by means of the bacterium Alcaligenes sp. 701B (Izumori et al., 1990).

Yoshihara et al. (2006) describe the direct conversion of D-psicose to D-tagatose (via D-talitol) using various fungi from the Mucoraceae family, such as Rhizopus oryzae MYA-2483.

However, this process is characterized by long reaction times (8 to 14 days) with only low titres of up to 3 g/l D-talitol and 4.2 g/l D-tagatose at a starting D-psicose concentration of 12 g/l. To ensure that the conversions are more economical, the authors propose screening for more effective strains, cultivation by mutation and genetic engineering, as well as optimization of the bioprocess.

Sasahara et al. (1998) also deal with the production of D-talitol from D-psicose. The reduction of D-psicose to D-talitol using the halotolerant yeast strain Candida famata R28 is described therein (as well as in JP 2020039301 A and JP 2006166718 A). The authors observed that the reduction can be accelerated in the presence of sugar alcohols such as D-sorbitol. However, the reaction rate is very slow so that the process requires 10 days to convert 10% D-psicose (100 g/l) to D-talitol by 95% in the presence of 5% D-sorbitol (50 g/l). When the amount of D-sorbitol used was halved, i.e., 25 g/l, a turnover of just about 55% could still be achieved after 10 days.

This is where the object of the present invention comes into play and aims to provide a biotechnological process for the production of D-talitol and of aqueous solutions of D-tagatose and/or D-psicose, which can be carried out more efficiently and, in particular, in a one-pot process.

### DETAILED SPECIFICATION OF THE INVENTION

The object of producing D-talitol is achieved according to the invention in that initially D-psicose is formed in vitro from D-fructose, which exists in a dissolved state in an aqueous solution, by treating it with an epimerase, whereupon the D-psicose is reduced in vitro to D-talitol by treating it with an NAD(P)H-dependent oxidoreductase, and the epimerase and the oxidoreductase are separated or deactivated.

D-Talitol can be concentrated and crystallized from the obtained solution.

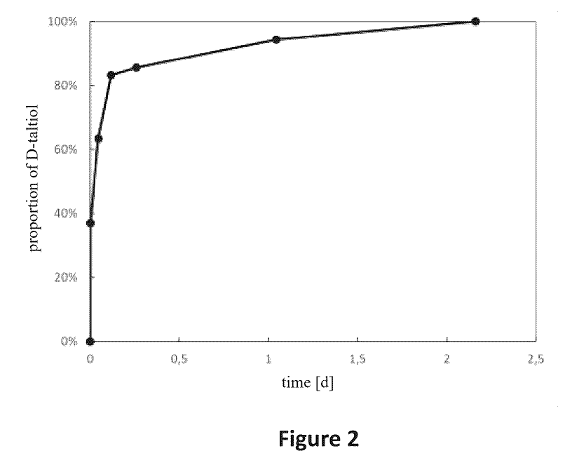

Surprisingly, it has been shown that the reduction of D-psicose to D-talitol can be accomplished in vitro much more efficiently than is known in the prior art (Sasahara et al., 1998). The D-talitol concentration over time is illustrated in the attached FIG. 2.

The object of producing an aqueous solution of D-tagatose or D-psicose is achieved in that the aqueous solution containing D-talitol, which has been prepared by the process according to the invention, is treated with an NAD(P)⁺-dependent oxidoreductase, thereby forming the reduced cofactor NAD(P)H, in order to oxidize D-talitol to D-tagatose or, respectively, D-talitol to D-psicose, whereupon the oxidoreductase is separated. Advantageously, these oxidations are carried out in vitro.

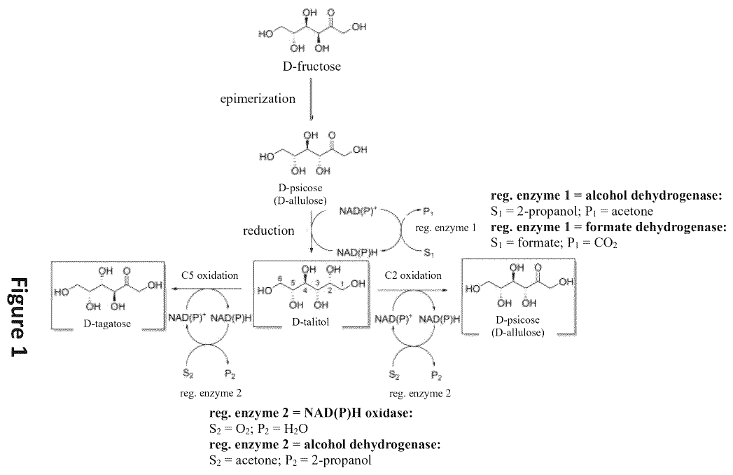

The reaction scheme of the processes according to the invention is depicted in the attached FIG. 1. In this context, the abbreviation “reg. enzyme” in FIG. 1 stands for regenerative enzyme.

In the case of the production of D-psicose, prior to the implementation of the final step (oxidation), the enzymes (epimerase and reductase/dehydrogenase) of the first step (D-fructose→D-talitol) are advantageously deactivated by the effect of heat or separated by ultrafiltration to prevent part of the D-psicose formed by the oxidation from being converted back to D-fructose by the epimerase.

A preferred embodiment of the process according to the invention for the production of an aqueous solution containing D-talitol consists in that the oxidized cofactor NAD(P)⁺ formed by the reduction is reduced by means of an alcohol dehydrogenase and a secondary alcohol, thereby forming a ketone, as described, for example, in EP 2812439 B1.

Furthermore, it has been shown that the secondary alcohol 2-propanol (isopropanol) is particularly suitable. 2-Propanol is a very favourable hydrogen donor for the regeneration of NAD(P)H, and the oxidation product acetone is readily separable due to its volatility (Xu et al., 2021). Acetone obtained from the waste gas stream can be hydrogenated back to 2-propanol in a heterogeneous/catalytic manner (Al-Rabiah et al., 2022), either in the gas phase, in solution, or in 2-propanol/acetone/water mixtures, with the future possibility of relying increasingly on hydrogen from sustainable sources (“green hydrogen”).

A further preferred embodiment of the process according to the invention for the production of an aqueous solution containing D-talitol consists in that the oxidized cofactor NAD(P)⁺ formed by the reduction is reduced by means of a formate dehydrogenase and a formic acid salt (formate), thereby forming carbon dioxide.

A preferred embodiment of this process according to the invention consists in that the reduced cofactor NAD(P)H formed by the oxidation is oxidized to NAD(P)⁺ by means of an oxidase and oxygen.

In a further preferred embodiment of this process according to the invention, the reduced cofactor NAD(P)H formed by the oxidation is oxidized to NAD(P)⁺ by means of NAD(P)-dependent alcohol dehydrogenase and a ketone, thereby forming a secondary alcohol.

Furthermore, it has been shown that acetone is particularly suitable as a ketone, with 2-propanol being formed as the reduction product.

For the regeneration of the cofactors required in the redox reactions, an NAD(P)-dependent alcohol dehydrogenase or an NAD(P)-dependent formate dehydrogenase is used in the case of the reduction of D-psicose to D-talitol, and an H₂O-forming NAD(P)H oxidase or an NAD(P)-dependent alcohol dehydrogenase is used in the case of the oxidation of the sugar alcohol to D-tagatose or D-psicose.

Another preferred variant of the processes according to the invention is characterized in that they are carried out as a one-pot reaction without isolation of intermediates.

The separation of the enzymes can be accomplished, for example, through heat deactivation and/or centrifugation or ultrafiltration.

The particularly preferred concentration of D-fructose is 50-250 g/l.

The particularly preferred temperature range for the first step (epimerization and reduction) is between 25 and 40° C., and for the second step (oxidation) between 2° and 35° C.

The particularly preferred pH range for the two steps is between 7.0 and 8.5.

In a further preferred variant of the process according to the invention, the enzymes are present in a suspension, in the homogenate and/or in the lysate of the corresponding cells producing them, with lysates being particularly preferred. In addition, the enzymes can also be used in a lyophilized or spray-dried or immobilized form in the process according to the invention. The enzymes used according to the invention can also be used in a substantially purified form so that the enzyme preparations will comprise no or substantially no components derived from the cells or, respectively, the culture in which they were expressed. Enzymes produced, for example, by recombinant expression (e.g., in E. coli) can be purified by methods known from the prior art in order to be used also in and for the production of food after, among other things, ultrafiltration and DNA removal.

Suspension in this context means a suspension of resting cells. These cells are harvested after cultivation (separated from the nutrient medium) and suspended in a suitable buffer system. In contrast to fermentative processes, which also work with whole cells, the resting cells can no longer grow due to the removal of carbon sources and nutrients, but only serve for the conversion of substrates (Lin & Tao, 2017). In this context, homogenate refers to a physically and/or chemically treated suspension (e.g., treated by pressure, lysozyme or ultrasound), whereby the cellular components are released from the cells. A lysate is obtained when the insoluble cellular components of the homogenate are removed, for example, by filtration or centrifugation (for details see Produktion der Enzyme & Herstellung der Lysate).

In a further variant, the enzymes can also be modified with a water-soluble polymer such as polyethylene glycol at the N-terminus, can be immobilized in or on a solid matrix, or can be part of a fusion protein.

In a further variant, the enzymes can be present in powder form, in a lyophilized form, or in a spray-dried form.

In a particularly preferred embodiment of the process, only enzymes from the enzyme groups of epimerases and oxidoreductases are used for the conversion of the starting material, with one or more of these enzymes being selected from each of these groups.

The epimerase used in the process may originate from one of the groups EC 5.1.3.30 (D-psicose 3-epimerase) or EC 5.1.3.31 (D-tagatose 3-epimerase/L-ribulose 3-epimerase), with the former being particularly preferred.

The NAD(P)H-dependent oxidoreductase for the reduction of D-psicose to D-talitol preferably comprises an amino acid sequence selected from the group consisting of:
i) an amino acid sequence having an identity to SEQ ID No. 2, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22 or SEQ ID No. 24 of at least 80%,
ii) an amino acid sequence encoded by a nucleic acid having an identity to SEQ ID No. 1, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21 or SEQ ID No. 23 of at least 80%, and
iii) an amino acid sequence encoded by a nucleic acid that binds under stringent conditions to a nucleic acid molecule having the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21 or SEQ ID No. 23.

SEQ ID No. 1:
ATGAACGATCCCGTTCTGGAAGCCAGACCGATTTTGGAGAGATTTCGCCT
CGACGGCCGCGTGGCGCTGGTGACCGGCGGTGGGCAGGGCATCGGCCGC
GCCTTCGCCCATGCGCTGTGCGAGGCAGGCGCCGCAGTGGCCGTCGTCGA
TCTGCGCCTGGACCTGGCCGAGGAAGTGGCGCATGAGCTGGTAAACAAG
CAGATCGACGCCATCGCAATTCAGGCCGATGTGACGAAGCCCGATCAGG
TGCAGACGATGGTTGATGCCATTCTGTCCAGGTGGGGGACGTTGACGATC
GGTGTCAACAACGCCGGCATCGGCTTGTGGGCGGATGCCGAAAGCATGG
CATACACAGACTGGCTGCGCGTGATCGATATTGATCTGAACTCGGTTTTC
CTCTGCGCCCAGGCTGAAGCGAGGGTCATGCTGCCGGCCGGCTACGGCA
AGATCATCAACACGGCGTCGATGTCCGGCCATATCAGCAACACGCCGCA
AAATCAGGCTGCTTACAACACGGCCAAGGCTGGCGTGATCCATCTGACGC
GCAGCCTTGCCGCGGAATGGGCCAAGCGGGGAGTGCGCGTCAACAGCAT
CAGCCCCGGCTATACGCGCACCAAATTGGTTGACGACCTGCTCGCCACGC
CGATCGGACAGACGATGTTGCCCACCTGGATGGGCATGACGCCCATGGG
ACGAATGGCCGAGGTCACCGATCTGCAAGGCGCCGTCGTCTACCTGGCGT
CGTCGGCGTCCGACTATATGACCGGCCATGACATGGTGATTGACGGCGGC
TATTGTTGCTGGTAA
SEQ ID No. 2:
MNDPVLEARPILERFRLDGRVALVTGGGQGIGRAFAHALCEAGAAVAVVDL
RLDLAEEVAHELVNKQIDAIAIQADVTKPDQVQTMVDAILSRWGTLTIGVN
NAGIGLWADAESMAYTDWLRVIDIDLNSVFLCAQAEARVMLPAGYGKIINT
ASMSGHISNTPQNQAAYNTAKAGVIHLTRSLAAEWAKRGVRVNSISPGYTR
TKLVDDLLATPIGQTMLPTWMGMTPMGRMAEVTDLQGAVVYLASSASDYMT
GHDMVIDGGYCCW
SEQ ID No. 5:
ATGTATCCAGATTTAAAAGGAAAAGTTGTCGCTATTACAGGAGCTGCTTC
AGGATTAGGGAAGGCAATGGCCATTCGCTTCGGCAAGGAGCAGGCAAAA
GTGGTTATCAACTACTACAGCAATAAGCAGGATCCGAACGAGGTAAAGG
AAGAGGTCATCAAGGCGGGCGGTGAAGCTGTTGTCGTCCAAGGAGACGT
AACAAAAGAGGAAGATGTAAAAAACATCGTCCAAACAGCGATTAACGAG
TTCGGTACACTCGATATTATGATTAATAATGCCGGTCTTGAAAATCCCGTT
CCTTCTCATGAAATGCCGCTGAAGGATTGGGATAAAGTAATCAGCACGAA
CTTAACGGGCGCCTTTTTAGGAAGCCGTGAAGCGATTAAATATTTTGTTG
AAAACGATATAAAAGGAAATGTCATTAATATGTCGAGCGTACATGAAGT
GATTCCGTGGCCATTATTTGTTCACTATGCGGCAAGTAAAGGCGGAATCA
AGCTGATGACGGAAACATTGGCGCTGGAATATGCGCCGAAAGGCATTCG
TGTCAACAATATCGGGCCAGGCGCGATCAACACGCCAATCAATGCTGAA
AAATTTGCTGATCCTAAGCAGAGAGCAGATGTAGAAAGCATGATTCCGAT
GGGATATATCGGTGAACCGGAGGAAATTGCGGCAGTAGCAGCCTGGCTT
GCTTCGAAGGAAGCCAGCTACGTCACAGGCATCACGTTATTCGCGGACGG
CGGTATGACCCAATATCCTTCCTTCCAGGCAGGACGCGGATAA
SEQ ID No. 6:
MYPDLKGKVVAITGAASGLGKAMAIRFGKEQAKVVINYYSNKQDPNEVKEE
VIKAGGEAVVVQGDVTKEEDVKNIVQTAINEFGTLDIMINNAGLENPVPSH
EMPLKDWDKVISTNLTGAFLGSREAIKYFVENDIKGNVINMSSVHEVIPWP
LFVHYAASKGGIKLMTETLALEYAPKGIRVNNIGPGAINTPINAEKFADPK
QRADVESMIPMGYIGEPEEIAAVAAWLASKEASYVTGITLFADGGMTQYPS
FQAGRG
SEQ ID No. 7:
ATGACTATTCCTAATACTACTGAAATTACCTCGTTTACAAACCCTGCTCTT
GGACCATTACCCACTTCAGCACCAGAACTAGCTACTAGTGTGCTTGATTT
ATTTCTGCTTAAGGGCAAAGTAGCATCAGTTACTGGATCCTCTACTGGTA
TAGGGTACGCTGTGGCAGAAGCTTTTGCACAGGCAGGTGCCGATGTTGCA
ATTTGGTACAACAATCATCCTGCAACCGAAAAGGCTGAAAAATTGGCTAA
AAAATATGGCGTTAAATGCAGGGCATACAAATGCAACATTTCCAATGCTG
ACGATGTTGATCTGACGATTAAGCAGATTGAGCAAGATTTTGGAACGATT
GATATCTTTGTTGCCAATGCTGGTGTGGCTTGGACTTCTGGTTCCATGGTT
GATGTGGACGATAATCATGAAAGTTGGCACAAGATTGTCGACTTGGATTT
GAATGGAGTATACTATTGTTGTCATTCAATAGGGAAAGTATTCAAGAGGA
AAGGAAAGGGTTCCTTGGTTATTACTTCATCAATGTCGGGCCTGATTGTC
AATGTTCCACAGCTACAGGCTCCTTATAATGCTGCTAAGGCTGCTGTAAA
ACATTTAGCTAAATCGTTAGCAGTTGAATGGGCTCCATTTGCTAGAGTCA
ATAGTATATCTCCAGGATATATCACTACTGATATTTCTGAATTCGCCGATC
CTGAAATGAAGGCAAGATGGTGGCAATTAACTCCTTTGGGGAGAGAGGG
ATTGCCACAAGAATTAGTAGGAGCTTACTTATACTTAGCCTCCAATGCCT
CTACATTTACTACGGGAGCCGATCTCGTTGTGGACGGTGGATACACTGCT
CCATAA
SEQ ID No. 8:
MTIPNTTEITSFTNPALGPLPTSAPELATSVLDLFLLKGKVASVTGSSTGI
GYAVAEAFAQAGADVAIWYNNHPATEKAEKLAKKYGVKCRAYKCNISNADD
VDLTIKQIEQDFGTIDIFVANAGVAWTSGSMVDVDDNHESWHKIVDLDLNG
VYYCCHSIGKVFKRKGKGSLVITSSMSGLIVNVPQLQAPYNAAKAAVKHLA
KSLAVEWAPFARVNSISPGYITTDISEFADPEMKARWWQLTPLGREGLPQE
LVGAYLYLASNASTFTTGADLVVDGGYTAP
SEQ ID No. 9:
ATGGAAAAACTCCGCCTCGATAACCGCGTCGCCATCGTCACCGGCGGCGC
ACAGAACATCGGCCTGGCCTGCGTCACCGCACTGGCCGAGGCTGGCGCCC
GCGTCATCATCGCCGATCTCGATGAAGCCATGGCCGCAAAAGCCGTGGA
AGATCTGCGCATGGAAGGCCATGATGTCAGCAGCGTCGTCATGGACGTCA
CGAACACGGAAAGTGTGCAGAACGCTGTCCGCAGCGTTCACGAGCAGGA
AGGCCGCGTGGATATCCTGGTCGCCTGCGCCGGCATCTGCATTTCCGAAG
TCAAGGCCGAGGACATGACGGATGGCCAGTGGCTCAAGCAGGTCGATAT
CAACCTGAACGGCATGTTCCGCTCCTGTCAGGCCGTCGGGCGCATCATGC
TCGAACAGAAACAGGGCGTCATTGTCGCAATCGGCTCCATGTCCGGCCTG
ATCGTCAACCGCCCGCAGCAGCAGGCGGCCTATAATGCGTCGAAAGCCG
GCGTTCACCAGTATATCCGCTCGCTCGCCGCAGAATGGGCTCCCCATGGC
ATTCGTGCCAACGCCGTTGCCCCCACCTATATCGAAACGACGCTCACCCG
CTTCGGCATGGAAAAGCCGGAACTCTACGACGCCTGGATCGCCGGAACC
CCGATGGGCCGCGTGGGCCAGCCCGATGAAGTCGCTTCCGTCGTGCAGTT
CCTGGCGTCCGACGCAGCCAGCCTGATGACCGGCGCCATCGTGAATGTGG
ATGCGGGCTTCACCGTCTGGTAA
SEQ ID No. 10:
MEKLRLDNRVAIVTGGAQNIGLACVTALAEAGARVIIADLDEAMAAKAVED
LRMEGHDVSSVVMDVTNTESVQNAVRSVHEQEGRVDILVACAGICISEVKA
EDMTDGQWLKQVDINLNGMFRSCQAVGRIMLEQKQGVIVAIGSMSGLIVNR
PQQQAAYNASKAGVHQYIRSLAAEWAPHGIRANAVAPTYIETTLTRFGMEK
PELYDAWIAGTPMGRVGQPDEVASVVQFLASDAASLMTGAIVNVDAGFTV
W
SEQ ID No. 11:
ATGTCTCAAGCTATCTCAGCAATCAACGAAAGAGTTGGCCCATTGCCCAC
TAAAGCACCCCAACTTTCAAAAAACGTATTAGACCTCTTTTCCTTGAAAG
GCAAGGTAGCATCGGTGACAGGTTCATCGAAGGGAATAGGCTTTGCTGTC
GCAGAAGCATACGCTCAAGCAGGGGCTGATGTTGCTTTGTGGTACAATTC
AAGTCCTGTCGAGGATAAGGTAAAATACTTGAAAGAGACCTACGGCGTG
AAAGTCAAAGCCTACAAGTGTAACGTGGGAAACAGCGAGGAGGTTGAGA
AAATAGTAGACCAAATTGCAGAGGACTTCGGCACAATTGACGTTTTTGTT
GCTAATGCGGGCATTGCTTGGAGCGAAGGTAGATCTCTCGAGGTTAAGGG
TTACGACGCTTGGCAAAAAATTGTCGACTACGACTTGAGCAGTGTATACT
ACTGTGCTAAGGCAGTGGGTAAGATCTTCCAAGAAAAGGGCTCCGGTTCG
TTAATCCTCACCGCATCCATGTCCGGACACATTGTGAATGTTCCTCAAATG
CAGGCACCATACAATGCGGCCAAGGCGGCTGTGATCCATCTAGGAAAAT
CTTTGGCTGTCGAATGGGCTCCATTTGCCAGGGTCAACACCGTTTCTCCTG
GCTATATTGCCACTGATATTGGTGAATTTGTGCTGCCCGATGAGAAGAAA
AAGTGGTGGTCATTGACACCCTTGGGTAGAGAAGGTCTTCCTCAAGAACT
CACTGGTGCATACTTGTATTTTGCTTCTAACGCATCGACCTACACTACTGG
GGCTGACTTGGTCGTAGACGGTGGCTACACTGTACCATAA
SEQ ID No. 12:
MSQAISAINERVGPLPTKAPQLSKNVLDLFSLKGKVASVTGSSKGIGFAVA
EAYAQAGADVALWYNSSPVEDKVKYLKETYGVKVKAYKCNVGNSEEVEKIV
DQIAEDFGTIDVFVANAGIAWSEGRSLEVKGYDAWQKIVDYDLSSVYYCAK
AVGKIFQEKGSGSLILTASMSGHIVNVPQMQAPYNAAKAAVIHLGKSLAVE
WAPFARVNTVSPGYIATDIGEFVLPDEKKKWWSLTPLGREGLPQELTGAYL
YFASNASTYTTGADLVVDGGYTVP
SEQ ID No. 13:
ATGTCACACCCAACATCAGTTATTAACGAGCAGGTAGGCCCATTACCAAC
TAAGGCCCCACAACTTTCTAAGAATGTGAATGATTTGTTCTCATTAAAGG
GTAAGGTAGCTTCTGTCACCGGTTCCTCAGGAGGGATTGGTTGGGCTGTA
GCTGAAGCGTATGCCCAAGCAGGAGCAGACGTTGCTGTTTGGTACAATTC
TAAAAATGCAGATGCTAAGGCTGAATACTTAACTAAGACTTACGGTGTGA
AGTCGAAGGCATACAAGTGTAACATTTCTGATCCAGAAGACGTCGAAAA
GGTAATTGGACAAATTGAAAAGGATTTCGGTACCATTGACGTTTTCGTTG
CCAATGCCGGTGTTCCATGGACAGAAGGTAGAAGTATTGAAGTTGAAGG
ATATGATTCGTGGAAGAAGGTTATAGATTTGGACTTGAGTGGTGTCTACT
ATTGCGCTAAGGCTGTCGGAAAGATTTTCAAGAAGAATGGTAAGGGTTCG
CTTGTGTTCACAGCATCAATGTCTGGCCACATTGTGAATGTTCCACAATTG
CAAGCTCCATACAACGCCGCAAAGGCCGGTGTTTTGCACTTGAGTAAGTC
ATTAGCTGTCGAATGGGCCCCATTCGCTAGAGTTAACACCATTTCACCTG
GTTATATTGCCACAGAAATTTCCGACTTCGTTCCGGACGATGTCAAGGCT
AAGTGGTGGCAATTAATTCCATTAGGAAGAGAAGCTCTTCCACAAGAATT
AGTTGGTGCTTACTTATATTTTGCATCCGATGCGTCTACTTACACTACAGG
ATCGGACTTATTAGTCGATGGTGGTTACTCTGCTCCATAA
SEQ ID No. 14:
MSHPTSVINEQVGPLPTKAPQLSKNVNDLFSLKGKVASVTGSSGGIGWAVA
EAYAQAGADVAVWYNSKNADAKAEYLTKTYGVKSKAYKCNISDPEDVEKVI
GQIEKDFGTIDVFVANAGVPWTEGRSIEVEGYDSWKKVIDLDLSGVYYCAK
AVGKIFKKNGKGSLVFTASMSGHIVNVPQLQAPYNAAKAGVLHLSKSLAVE
WAPFARVNTISPGYIATEISDFVPDDVKAKWWQLIPLGREALPQELVGAYL
YFASDASTYTTGSDLLVDGGYSAP
SEQ ID No. 15:
ATGGGCAAGACTGTAGCAACTGGTATTGAAACCCCCCAACCTTATCCACA
ATTGCCTGAACATGTTATGGACATGTTTTCATTAAAAGGTAAGGTGGCCT
CTGTGACTGGTGCTTCCGGTGGTATTGGTTATGAAGTGGCCGTTGCATTTG
CACAGGCAGGTGCCAATGTTGCTATGTGGTACAACTCGCATTCTGTCGAG
GAGGAAGCTGAAAAGCTGTCGAAGAAATACAACGTCACCGTTAAGGCTT
ATAAATGTTCATTGACTGATACTAAGGCTGTTGAAGAAACTGTGCAACAG
ATTAAGAAGGACTTTGGTGGTAGAATTGATATAATGGTGGCTAATGCTGG
TGTGGCGTGGGATAAGGGTCCCTTGACCGAGCTGGCCGAAAAAGATTCTG
AGCTTTGTGACAAGGAATGGCAGAAAGTTTTGAGCATCGATATCAATGGT
GTCTATAATGCAGCCAAGAGTATTGGTCCAATTTTCAAGAAGCAAGGATC
AGGTTCTTTCATTGCAACTGGTTCGATGTCTGGTCACATTGCAAACGTTCC
GCAATTACAGGTCGCATACAATACTGCAAAGGCTGCCGTTATTCATATGT
GCAAATCGCTGGCTGTCGAATGGACAGGTTACGCCCGTGCTAACACTGTA
TCCCCAGGTTACGTAGCCACTCCATTGAATGCTGGGATGGACGAAGAGAT
GCTAAAGAAATGGAACACCTTAGTCCCACTAGGTCGTCTCGCTTTGCCCA
AGGAAATGGTTGGTGCTTATTTGTACTTGGCCTCTAATGCCTCAACATAC
ACGACCGGTAGCGACATACTAGTGGATGGTGGTTACTGTAGTGTTTAA
SEQ ID No. 16:
MGKTVATGIETPQPYPQLPEHVMDMFSLKGKVASVTGASGGIGYEVAVAFA
QAGANVAMWYNSHSVEEEAEKLSKKYNVTVKAYKCSLTDTKAVEETVQQI
KKDFGGRIDIMVANAGVAWDKGPLTELAEKDSELCDKEWQKVLSIDINGVY
NAAKSIGPIFKKQGSGSFIATGSMSGHIANVPQLQVAYNTAKAAVIHMCKS
LAVEWTGYARANTVSPGYVATPLNAGMDEEMLKKWNTLVPLGRLALPKEM
VGAYLYLASNASTYTTGSDILVDGGYCSV
SEQ ID No. 17:
ATGGTTGATAAGATTCTGGATAGGTTCAGTTTAAAAGGGAAAACCGCATT
GGTGACAGGTGGTGGTCAGGGTATTGGACGCGGATATGCTTTTGCGCTGG
GAGAAGCCGGGGCCAAGGTAGCCATTGTTGATATCAATGGAGAAACTGC
TGAAGCGACAGCCAAGGACCTGCAAGCAGCGGGGATTGCATCAATGTCC
TATGTCTGTGATGTAACTAATCCCGATCAGGTGATGGCAATGGTGAAACA
TATTGTCAAAGAATGGGGGACTTTGACTATAGGGGTTAACAACGCTGGCA
TGGGAATCTGGAGCGATGCAGTAACCATGCCCTATGAGATGTGGAGAAA
GACCATGGCTTTAAACATGGATGGGATTTTTCTTTGTGCCAGGGAGGAGG
CAAAGTATATGATACGCGAGGGGTATGGGAAAATTATCAATACTGCTTCG
ATGAGCGCCCATATATCGAATACCCCACAGAATCAAGTAGCTTACAATGC
ATCCAAGGCGGGGGTTCTCCACATGACCAGATCTTTGGCCGCAGAATGGG
CTCCCTATGGCGTGAGGGTGAACTCCATCAGTCCCGGATATACAAGGACA
GAGCTGGTCGAAAAGCTGCTTGCAACTCCAGAGGGGAAAAAAATGGAAT
CAGACTGGTTGCAACTGATTCCCCAAAAAAAGATGGCTACCGTCGAGGAT
TTGCAAGGCGCTTGCTTGTACCTTGCCTGCAGGGCTTCGGATTACACAAC
AGGTAGCGATATTCTCATAGATGGTGGGTATTGCTGTTGGTAA
SEQ ID No. 18:
MVDKILDRFSLKGKTALVTGGGQGIGRGYAFALGEAGAKVAIVDINGETAE
ATAKDLQAAGIASMSYVCDVTNPDQVMAMVKHIVKEWGTLTIGVNNAGM
GIWSDAVTMPYEMWRKTMALNMDGIFLCAREEAKYMIREGYGKIINTASMS
AHISNTPQNQVAYNASKAGVLHMTRSLAAEWAPYGVRVNSISPGYTRTELV
EKLLATPEGKKMESDWLQLIPQKKMATVEDLQGACLYLACRASDYTTGSDI
LIDGGYCCW
SEQ ID No. 19:
ATGGCAGAAAAAATTCTTGACAGATTCAAGCTGGACGGCAAGACCGCCC
TGGTTACCGGAGGAGGGCAGGGCATTGGCCAGGCCTACTGTTTCGCCCTG
GGCGAAGCAGGCGCAAAGATAGCTGTGGTAGACATCAACACCACCGTTG
CGGAAGAGACCGCCCAGGCCCTGACAAAAAAAGGCATAGAGGCTATTGC
GATAACCACAGATGTTACAAAAGAAGACGAAGTAATAAAGATGGTTAAA
ACCGTCATAGACAAATGGGGCTTCCTCACCATAGGCGTCAACAACGCCGG
CATGGGTGTCTGGCGCGATGCGTTAACCCAGGACTTTGCCGAATGGCGGA
AGATCCTCTCCCTCAACCTCGATTCGATCTTCCTCTGCTCTCGCACCGAAG
CCGTGGAAATGGCAAAGAAAGGCTATGGCAAGATCGTCAACACCGCTTC
CATGTCCGCCCATATTTCCAACACCCCCCAGAACCAGGCAGCCTACAACA
GCTCCAAAGCCGGTGTGCTCCATCTGACCCGCAGCCTCGCTGCCGAATGG
GCGCCCAAAAATATCAGGGTAAACAGCATCTCCCCGGGTTATACAAAAA
CCGCCCTGGTAGACAAGCTCCTCGAAACCCCGGAAGGCAAAACCATGCT
GCCCAAGTGGCTGGAAAAAGTCCCCATGGGGCGCATGGCTACCGTGGAA
GATCTGCAAGGCGCCGTTGTGTATCTGGCATCGCCCGCATCGGATTATGC
CACCGGCACAGATATTATCATTGACGGCGGATATTGCTGCTGGTAA
SEQ ID No. 20:
MAEKILDRFKLDGKTALVTGGGQGIGQAYCFALGEAGAKIAVVDINTTVAE
ETAQALTKKGIEAIAITTDVTKEDEVIKMVKTVIDKWGFLTIGVNNAGMGV
WRDALTQDFAEWRKILSLNLDSIFLCSRTEAVEMAKKGYGKIVNTASMSAH
ISNTPQNQAAYNSSKAGVLHLTRSLAAEWAPKNIRVNSISPGYTKTALVDK
LLETPEGKTMLPKWLEKVPMGRMATVEDLQGAVVYLASPASDYATGTDIII
DGGYCCW
SEQ ID No. 21:
ATGGTTCTCTCTCAGCCCGAAAACAAGCATGTTATGAAAGCCTTCGATTT
GACCGGCAAGGTCGCAGCTGTGACAGGAGGAGCTCGCGGGATCGGTCTT
GAGGTCTCCAGAGGGCTTGCAGAAGCTGGTGCGAATGTTGCAGTCATCTA
CAGCTCGTCCAAGAATGCCGACGCCGTTGCAGCCGAGATCGCAGCTGCCA
ACAACGTCAAGACAGCCGCATACAAGGCAGATGTAAGCAACCAGCAGGA
TATCGAGAGCACCATTCAGCAGATAGCAAAGGACTTTGGAAAACTGGAC
ATAATCGTGGCGAATTCTGGAATCGCAAGCACGCATCCCGCTGAAGACTA
CACAGTGGAGGAGTGGAGAGATATCCTCAAAGTGAATCTAGATGGAGCG
TTCTACACAGCGCAGGCGGCGGCAAGGATCTTCAAGACGCAAGGGCATG
GAAACGTCATCTTCACGGCTTCTGTCAGCGCAAGGCTGGTCAATGTCCCC
CAGAAACAAGCTGCGTACAACGCCTCGAAAGCTGGTCTTGTCCAGCTGGC
AAAGTGCTTGTCAGTGGAATGGATAGACTATTGTCGTGTCAACTGCATTT
CGCCTGGTTTTATTGCGACAGAAATCTTGGATATCCACCCCAAGGAGTGG
AGAGAAAAATGGCTCGACATGGTTCCCGCGCGTCGGATGGCTGCTACTTA
CGAGCTAAAGGGGGCGTATGTTTTCTGTGCTTCTGACGCGTCAAGCTACA
TGACCGGCGCTGATATAGTCATTGATGGTGGATATACGCTTCCGTAA
SEQ ID No. 22:
MVLSQPENKHVMKAFDLTGKVAAVTGGARGIGLEVSRGLAEAGANVAVIY
SSSKNADAVAAEIAAANNVKTAAYKADVSNQQDIESTIQQIAKDFGKLDII
VANSGIASTHPAEDYTVEEWRDILKVNLDGAFYTAQAAARIFKTQGHGNVI
FTASVSARLVNVPQKQAAYNASKAGLVQLAKCLSVEWIDYCRVNCISPGFI
ATEILDIHPKEWREKWLDMVPARRMAATYELKGAYVFCASDASSYMTGADI
VIDGGYTLP
SEQ ID No. 23:
ATGGCTGCGTCTCCCATCACCAACGGCCTCTTCAACCATGACAACTCCAC
CCCGCCCGAGCATCCCAGCCTGTTCGCGCTGTTCTCGCTCAAGGGCAAGA
CCGCCATCGTCACGGGCGCCGGTGCTGGCATCGGCCTGCATGTGGCTCAT
GGTCTGGCTGAGGCTGGCGCCAACGTTGCGCTCTTCTACAACACCAACAC
CAAGACGCCGGAGCGCGCTGCCGAGATCGAGCAGCAGTATGGCGTGAAG
GCCAAGGCATACCAGGTCGACGTCCGCGACGCCAAGAAGCTCGAGGAGA
CGGTCAACCAGGCCGTGCGCGACCTCAACGGCCGCCTGGACATCTTCATC
GCCAACGCCGGCATCCCGTGGACCAAGGGCCCCAGCGTTGACGGCCCGC
TGGACCACTACCAGGCCGTCGTGCAGACCGACCTGGACGGCGTCTTCTAC
TCGGCCAAGGCGGCGGCAGCCCACTGGCGGCGCCAGAAGGAGGAGGGCA
CCGACCTGTTCGGCAACAAGCTGCAGAACTTCACCTACGGCAGCTTCGTG
GCCACGGCGTCGATGAGCGGCCATATCGTGAACATTCCCCAGCTGCAGGC
GGCGTACAACGCCGCCAAGGCGGGCGTCATCCACATGGTCAAATCGTTTG
CCGTCGAATGGGCGCGCTTCGCCCGTGCCAACTCGGTCTCTCCTGGCTAC
ATCGCCACGGAGATCTCCAACTTCGTTCCCGCGGAGACCAAGAAGCTCTG
GCGGGACAAGACGCCGCTGGGCCGCGAGGGTCTGCCGCAGGAGCTCAAG
GGCGCCTACCTGTACCTGGCCAGCGATGCGGCCAGCTTCACGACGGGCGC
GGACCTGGTGGTCGACGGAGGCTACACCCTGCCTTAA
SEQ ID No. 24:
MAASPITNGLFNHDNSTPPEHPSLFALFSLKGKTAIVTGAGAGIGLHVAHG
LAEAGANVALFYNTNTKTPERAAEIEQQYGVKAKAYQVDVRDAKKLEETVN
QAVRDLNGRLDIFIANAGIPWTKGPSVDGPLDHYQAVVQTDLDGVFYSAKA
AAAHWRRQKEEGTDLFGNKLQNFTYGSFVATASMSGHIVNIPQLQAAYNAA
KAGVIHMVKSFAVEWARFARANSVSPGYIATEISNFVPAETKKLWRDKTPL
GREGLPQELKGAYLYLASDAASFTTGADLVVDGGYTLP

Surprisingly, it has been shown that an oxidoreductase having one of the amino acid sequences SEQ ID No. 2, SEQ ID No. 6, SEQ ID No. 8, SEQ ID No. 10, SEQ ID No. 12, SEQ ID No. 14, SEQ ID No. 16, SEQ ID No. 18, SEQ ID No. 20 is not only capable of reducing D-psicose to D-talitol, which reaction can preferably take place in the presence of NAD(P)H, but also of oxidizing D-talitol to D-psicose, which reaction can preferably take place in the presence of NAD(P)⁺.

The oxidoreductase for the formation of D-talitol from D-psicose and, respectively, of D-psicose from D-talitol preferably comprises an amino acid sequence having an identity to SEQ ID No. 2, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22 or SEQ ID No. 24 of at least 80%, more preferably of 85%, more preferably of 90%, more preferably of 95%, more preferably of 98%, more preferably of 99%, in particular of 100%. Particularly preferably, the oxidoreductase according to the invention for the formation of D-talitol from D-psicose comprises or consists of the amino acid sequence SEQ ID No. 2, SEQ ID No. 6, SEQ ID No. 8, SEQ ID No. 10, SEQ ID No. 12, SEQ ID No. 14, SEQ ID No. 16, SEQ ID No. 18 or SEQ ID No. 20.

Alternatively, the oxidoreductase for the formation of D-talitol from D-psicose and, respectively, of D-psicose from D-talitol preferably comprises an amino acid sequence encoded by a nucleic acid having an identity to SEQ ID No. 1, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21 or SEQ ID No. 23 of at least 80%, more preferably of 85%, more preferably of 90%, more preferably of 95%, more preferably of 98%, more preferably of 99%, in particular of 100%. Particularly preferably, the nucleic acid encoding the oxidoreductase according to the invention for the formation of D-talitol from D-psicose comprises or consists of the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17 or SEQ ID No. 19.

The term “identity”, as used herein, refers to the percentage of identical nucleotide or amino acid matches between at least two nucleotide or amino acid sequences aligned to each other using a standardized algorithm (“alignment”). Such an algorithm can insert “gaps” into the compared sequences in a standardized and reproducible manner in order to optimize the alignment between two sequences, thus obtaining a more meaningful comparison of the two sequences.

The percentage identity between sequences can be determined using one or more computer algorithms or programs known from the prior art or described herein. According to the invention, the Basic Local Alignment Search Tool (BLAST) (Altschul et al., 1990) provided by the National Center for Biotechnology Information (NCBI) is used for the determination of identity. The BLAST software suite contains various programs, including a tool called “BLAST 2 Sequences,” which is used for the direct pairwise comparison of two nucleotide or amino acid sequences. “BLAST 2 Sequences” can also be accessed and used interactively via the NCBI World Wide Web page on the internet. The blastn program (for nucleotide sequences) uses a word length (W) of 11, an expectation (E) of 10, M=5, N=−4, and a comparison of the two strands as defaults. For amino acid sequences, the blastp program uses a word length of 3 and an expectation (E) of 10 as defaults, and the BLOSUM62 scoring matrix (Henikoff & Henikoff, 1989), alignments (B) of 50, expectation (E) of 10, M=5, N=−4.

Alternatively, the oxidoreductase for the formation of D-talitol from D-psicose and, respectively, of D-psicose from D-talitol preferably comprises an amino acid sequence encoded by a nucleic acid that binds under stringent conditions to a nucleic acid molecule having the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21 or SEQ ID No. 23. As used herein, stringent conditions refer to conditions under which so-called specific hybrids, but no non-specific hybrids, are formed. For example, stringent conditions include hybridization in 6×SSC (sodium chloride/sodium citrate) at 45° C. followed by washing with 0.2 to 1×SSC, 0.1% SDS at 50 to 65° C.; or such conditions may include hybridization in 1×SSC at 65 to 70° C. followed by washing with 0.3×SSC at 65 to 70° C.

Hybridization can be performed by commonly known methods, such as those described by J.

Sambrook et al. in Molecular Cloning, A Laboratory Manual, 2nd Ed., Cold Spring Harbor Laboratory (1989).

A further aspect of the present invention relates to a process for the production of D-talitol or D-psicose, comprising the step of treating D-psicose or, respectively, D-talitol with an oxidoreductase comprising an amino acid sequence selected from the group consisting of:
i) an amino acid sequence having an identity to SEQ ID No. 2, SEQ ID No. 6, SEQ ID No. 8, SEQ ID No. 10, SEQ ID No. 12, SEQ ID No. 14, SEQ ID No. 16, SEQ ID No. 18 or SEQ ID No. 20 of at least 80%,
ii) an amino acid sequence encoded by a nucleic acid having an identity to SEQ ID No. 1, SEQ ID No. 5, SEQ ID No. 7, SEQ ID No. 9, SEQ ID No. 11, SEQ ID No. 13, SEQ ID No. 15, SEQ ID No. 17 or SEQ ID No. 19 of at least 80%, and
iii) an amino acid sequence encoded by a nucleic acid that binds under stringent conditions to a nucleic acid molecule having the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17 or SEQ ID No. 19.

A further aspect of the present invention relates to a process for the production of D-talitol, comprising the step of treating D-psicose with an oxidoreductase comprising an amino acid sequence selected from the group consisting of:
i) an amino acid sequence having an identity to SEQ ID No. 22 or SEQ ID No. 24 of at least 80%,
ii) an amino acid sequence encoded by a nucleic acid having an identity to SEQ ID No. 21 or SEQ ID No. 23 of at least 80%, and
iii) an amino acid sequence encoded by a nucleic acid that binds under stringent conditions to a nucleic acid molecule having the nucleic acid sequence SEQ ID No. 21 or SEQ ID No. 23.

Yet another aspect of the present invention relates to the use of an oxidoreductase for the reduction of D-psicose to D-talitol and/or for the oxidation of D-talitol to D-psicose comprising an amino acid sequence selected from the group consisting of:
i) an amino acid sequence having an identity to SEQ ID No. 2, SEQ ID No. 6, SEQ ID No. 8, SEQ ID No. 10, SEQ ID No. 12, SEQ ID No. 14, SEQ ID No. 16, SEQ ID No. 18 or SEQ ID No. 20 of at least 80%,
ii) an amino acid sequence encoded by a nucleic acid having an identity to SEQ ID No. 1, SEQ ID No. 5, SEQ ID No. 7, SEQ ID No. 9, SEQ ID No. 11, SEQ ID No. 13, SEQ ID No. 15, SEQ ID No. 17 or SEQ ID No. 19 of at least 80%, and
iii) an amino acid sequence encoded by a nucleic acid that binds under stringent conditions to a nucleic acid molecule having the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17 or SEQ ID No. 19.

A further aspect of the present invention relates to the use of an oxidoreductase for the reduction of D-psicose to D-talitol comprising an amino acid sequence selected from the group consisting of:
i) an amino acid sequence having an identity to SEQ ID No. 22 or SEQ ID No. 24 of at least 80%,
ii) an amino acid sequence encoded by a nucleic acid having an identity to SEQ ID No. 21 or SEQ ID No. 23 of at least 80%, and
iii) an amino acid sequence encoded by a nucleic acid that binds under stringent conditions to a nucleic acid molecule having the nucleic acid sequence SEQ ID No. 21 or SEQ ID No. 23.

The NAD(P)⁺-dependent oxidoreductase for the oxidation of D-talitol to D-tagatose preferably comprises an amino acid sequence selected from the group consisting of:
i) an amino acid sequence having an identity to SEQ ID No. 4 of at least 80%,
ii) an amino acid sequence encoded by a nucleic acid having an identity to SEQ ID No. 3 of at least 80%, and
iii) an amino acid sequence encoded by a nucleic acid that binds under stringent conditions to a nucleic acid molecule having the nucleic acid sequence SEQ ID No. 3.

SEQ ID No. 3:
ATGGCTGCCAACGTCCCCAAGACCATGAAGGCTCTCAGATATGAGAAGC
CTGAGACGTTCTCCATCGTCGACATTCCCGTTCCCACTCTGCGTGAGAAC
GATGTCTTGATCAAGGTCAAGGCTTGCGGTGTCTGTGGTACCGACCTGCA
CATTCACGAGGGAGAATTCCTTGCCAAGTTCCCTCTCGTTCCTGGCCACG
AGACTGTCGGTGTTGTTGCCGCAGTTGGACCCAAGGTCAAGGGTTTCGAG
ATCGGTGACCGTGTTGTTGCCGACAACTCCGAGCTTTGCGGCCAATGCTT
CTACTGCCGACGAGGAGAGGAGTTGCTCTGCGAGCACTTCGAAGCTCACG
GTGTCACGATGAACGGCGGTTTCGCTGAGTACTGCGCCTACCCTGCCGGC
CGTGTCTTCAAGATCAAGAACCTCTCTGACGTGGACGCCACTCTGCTTGA
GCCCGCGTCCTGCGCCGCTCACGGTCTGGACAAGATTGCCCCCAAGATGG
GCTCGTCCGTCCTGGTGTTCGGCGCCGGTCCCACCGGTCTGGTCCTTGCTC
AGATGCTCCGTCTGAACGGAGGATGCCGCGTCGTCGTCGCTGCGCCCGAG
GGTCTGAAGATGGACCTGGCCCAGAAGCTCGGCGCTGGTGATGAATACG
TTGCTCTTTCTCGCACGAACCCTCAGGCTCAGTTTGACAAGCTGAAGGCC
GACAACCCGTACGGCTTCGACATTGTCGTCGAGGCTACCGGCAATGCCAA
GATCCTGGAGGATGCCATCAACTATGTCCGCCGTGGAGGCAAACTGGTCG
TGTACGGTGTGTACGCGAACAAGGACCGCGTCTCGTGGCCCCCGAGCAA
GATCTTCGGTGACGAAATCACCATTCTGGGTAGCTTCTCCGAGACCTACA
AGTTCCCCGCCGCCATCGACTACCTGGACTCCGGCAAGGTGAAGGTCCAG
GGCATCGTGAACAAGACCTTCCGGCTGGAGCAGTGGGAGGAGTGTCTGG
CGTCGTTGAAGAACAAGAGCGCCATCAAGGCGGCGATCGTCTTTGACTAA
SEQ ID No. 4:
MAANVPKTMKALRYEKPETFSIVDIPVPTLRENDVLIKVKACGVCGTDLHI
HEGEFLAKFPLVPGHETVGVVAAVGPKVKGFEIGDRVVADNSELCGQCFYC
RRGEELLCEHFEAHGVTMNGGFAEYCAYPAGRVFKIKNLSDVDATLLEPAS
CAAHGLDKIAPKMGSSVLVFGAGPTGLVLAQMLRLNGGCRVVVAAPEGLKM
DLAQKLGAGDEYVALSRTNPQAQFDKLKADNPYGFDIVVEATGNAKILEDA
INYVRRGGKLVVYGVYANKDRVSWPPSKIFGDEITILGSFSETYKFPAAID
YLDSGKVKVQGIVNKTFRLEQWEECLASLKNKSAIKAAIVFD

Surprisingly, it has been shown that an oxidoreductase having one of the above-mentioned amino acid sequences is capable of oxidizing D-talitol to D-tagatose, which reaction can preferably take place in the presence of NAD(P)⁺.

The oxidoreductase for the formation of D-tagatose from D-talitol preferably comprises an amino acid sequence having an identity to SEQ ID No. 4 of at least 80%, more preferably of 85%, more preferably of 90%, more preferably of 95%, more preferably of 98%, more preferably of 99%, in particular of 100%. Particularly preferably, the oxidoreductase according to the invention for the formation of D-tagatose from D-talitol comprises or consists of the amino acid sequence SEQ ID No. 4.

Alternatively, the oxidoreductase for the formation of D-tagatose from D-talitol preferably comprises an amino acid sequence encoded by a nucleic acid having an identity to SEQ ID No. 3 of at least 80%, more preferably of 85%, more preferably of 90%, more preferably of 95%, more preferably of 98%, more preferably of 99%, in particular of 100%. Particularly preferably, the nucleic acid encoding the oxidoreductase according to the invention for the formation of D-tagatose from D-talitol comprises or consists of the nucleic acid sequence SEQ ID No. 3.

Alternatively, the oxidoreductase for the formation of D-tagatose from D-talitol preferably comprises an amino acid sequence encoded by a nucleic acid that binds under stringent conditions (see the above definition) to a nucleic acid molecule having the nucleic acid sequence SEQ ID No. 3.

A further aspect of the present invention relates to a process for the production of D-tagatose, comprising the step of treating D-talitol with an oxidoreductase comprising an amino acid sequence selected from the group consisting of:
i) an amino acid sequence having an identity to SEQ ID No. 4 of at least 80%,
ii) an amino acid sequence encoded by a nucleic acid having an identity to SEQ ID No. 3 of at least 80%, and
iii) an amino acid sequence encoded by a nucleic acid that binds under stringent conditions to a nucleic acid molecule having the nucleic acid sequence SEQ ID No. 3.

Yet another aspect of the present invention relates to the use of the oxidoreductase for the oxidation of D-talitol to D-tagatose comprising an amino acid sequence selected from the group consisting of:
i) an amino acid sequence having an identity to SEQ ID No. 4 of at least 80%,
ii) an amino acid sequence encoded by a nucleic acid having an identity to SEQ ID No. 3 of at least 80%, and
iii) an amino acid sequence encoded by a nucleic acid that binds under stringent conditions to a nucleic acid molecule having the nucleic acid sequence SEQ ID No. 3.

The alcohol dehydrogenase (ADH) used for cofactor regeneration can originate from one of the groups EC 1.1.1.1 (NAD-dependent ADH) and EC 1.1.1.2 (NADP-dependent ADH).

The NAD(P)⁺ forming during the reduction of D-psicose to D-talitol is reduced to NAD(P)H by means of formate and a formate dehydrogenase, thereby forming CO₂ (cofactor regeneration).

Particularly preferably, a formate dehydrogenase comprising or consisting of the amino acid sequence SEQ ID No. 26 or a functional fragment of this formate dehydrogenase is used. The formate dehydrogenase that is used preferably is preferably encoded by the nucleic acid sequence SEQ ID No. 25. A “functional fragment” of the formate dehydrogenase comprises an N-terminally and/or C-terminally truncated variant of the formate dehydrogenase having the amino acid sequence SEQ ID No. 26, which has at least 50%, preferably at least 60%, more preferably at least 70%, more preferably at least 80%, more preferably at least 90%, more preferably at least 95%, enzyme activity compared to the non-truncated formate dehydrogenase.

SEQ ID No. 25:
ATGGCGAAAATACTTTGCGTTCTCTATGACGATCCGGTCGACGGCTACCC
GAAGACCTATGCGCGCGACGACCTGCCGAAGATCGACCACTATCCGGGC
GGGCAGACGCTGCCCACGCCCAAGGCGATCGACTTCACGCCGGGCGCGC
TGCTCGGCTCGGTCTCCGGCGAGCTCGGCCTGCGCAAATACCTGGAAGCC
AACGGCCATACCTTCGTCGTCACCTCCGATAAGGACGGCCCGGATTCGGT
GTTCGAGAGGGAACTCGTCGACGCCGACGTGGTGATCTCGCAGCCCTTCT
GGCCGGCCTATCTGACGCCCGAGCGCATCGCCAAGGCGAAGAACCTGAA
GCTCGCGCTCACCGCCGGCATCGGCTCCGATCATGTCGATCTTCAGTCAG
CTATCGACCGTGGCATCACTGTGGCCGAAGTCACATATTGCAACTCGATC
AGCGTCGCCGAGCACGTGGTGATGATGATCCTCGGCCTGGTACGAAACTA
CATTCCCTCGCATGACTGGGCGCGCAAGGGCGGCTGGAACATAGCCGACT
GCGTAGAGCACTCCTACGACCTCGAGGGCATGACCGTCGGCTCGGTGGCC
GCCGGCCGCATCGGCCTCGCCGTGCTGCGCCGCCTCGCGCCGTTCGACGT
GAAGCTGCACTATACCGACCGCCACCGTCTGCCAGAAGCGGTCGAGAAG
GAGCTGGGCCTCGTCTGGCACGATACCCGCGAGGACATGTACCCGCATTG
CGACGTGGTCACGCTCAACGTGCCGCTGCACCCCGAAACCGAGCACATG
ATCAATGACGAGACGCTGAAGCTGTTCAAGCGCGGCGCCTATATCGTCAA
CACCGCCCGCGGCAAGCTCGCCGACCGCGACGCCATCGTCCGCGCGATCG
AGAGCGGGCAGCTCGCGGGCTATGCCGGCGACGTGTGGTTCCCGCAGCC
GGCTCCGAAGGACCACCCCTGGCGCACCATGAAGTGGGAAGGCATGACG
CCGCACATCTCCGGCACCTCGCTCTCTGCCCAGGCGCGCTACGCGGCGGG
CACGCGCGAGATCCTCGAATGCTTCTTCGAGGGCCGGCCGATCCGCGACG
AGTACCTGATCGTGCAGGGCGGCGCGCTCGCCGGCACCGGCGCGCATTCC
TACTCGAAGGGCAATGCGACCGGCGGTTCGGAAGAGGCCGCGAAGTTCA
AGAAGGCTGGCTGA
SEQ ID No. 26:
MAKILCVLYDDPVDGYPKTYARDDLPKIDHYPGGQTLPTPKAIDFTPGALL
GSVSGELGLRKYLEANGHTFVVTSDKDGPDSVFERELVDADVVISQPFWPA
YLTPERIAKAKNLKLALTAGIGSDHVDLQSAIDRGITVAEVTYCNSISVAE
HVVMMILGLVRNYIPSHDWARKGGWNIADCVEHSYDLEGMTVGSVAAGRIG
LAVLRRLAPFDVKLHYTDRHRLPEAVEKELGLVWHDTREDMYPHCDVVTLN
VPLHPETEHMINDETLKLFKRGAYIVNTARGKLADRDAIVRAIESGQLAGY
AGDVWFPQPAPKDHPWRTMKWEGMTPHISGTSLSAQARYAAGTREILECFF
EGRPIRDEYLIVQGGALAGTGAHSYSKGNATGGSEEAAKFKKAG

According to a further preferred embodiment of the present invention, the formate dehydrogenase used for cofactor regeneration comprises an amino acid sequence selected from the group consisting of:
i) an amino acid sequence having an identity to SEQ ID No. 26 of at least 80%,
ii) an amino acid sequence encoded by a nucleic acid having an identity to SEQ ID No. 25 of at least 80%, and
iii) an amino acid sequence encoded by a nucleic acid that binds under stringent conditions to a nucleic acid molecule having the nucleic acid sequence SEQ ID No. 25.

The formate dehydrogenase preferably comprises an amino acid sequence having an identity to SEQ ID No. 26 of at least 80%, more preferably of 85%, more preferably of 90%, more preferably of 95%, more preferably of 98%, more preferably of 99%, in particular of 100%.

Alternatively, the formate dehydrogenase preferably comprises an amino acid sequence encoded by a nucleic acid having an identity to SEQ ID No. 25 of at least 80%, more preferably of 85%, more preferably of 90%, more preferably of 95%, more preferably of 98%, more preferably of 99%, in particular of 100%.

The NAD(P)H oxidase used for cofactor regeneration can originate from one of the groups EC 1.6.3.1 (NAD(P)H oxidase (H₂O₂-forming)), EC 1.6.3.2 (NAD(P)H oxidase (H₂O-forming)), EC 1.6.3.3 (NADH oxidase (H₂O₂-forming)) and EC 1.6.3.4 (NADH oxidase (H₂O-forming)), with the H₂O-forming classes being particularly preferred.

Preferably, an H₂O-forming NAD(P)H oxidase that is used particularly preferably comprises or consists of an amino acid sequence selected from the group consisting of:
i) an amino acid sequence having an identity to SEQ ID No. 28, SEQ ID No. 30 or SEQ ID No. 32 of at least 80%,
ii) an amino acid sequence encoded by a nucleic acid having an identity to SEQ ID No. 27, SEQ ID No. 29 or SEQ ID No. 31 of at least 80%, and
iii) an amino acid sequence encoded by a nucleic acid that binds under stringent conditions to a nucleic acid molecule having the nucleic acid sequence SEQ ID No. 27, SEQ ID No. 29 or SEQ ID No. 31.

SEQ ID No. 27:
ATGAAAGTAGTAGTAGTAGGCTGTACACATGCAGGAACAGCGGCAGTTA
AGACGATTTTAAATGAACATCCAGATGCATCAGTATCAGTATATGAGCGT
AATGACAATGTCTCATTTCTATCTTGTGGGATTGCGTTGTATGTTGGTGGA
GTTGTGAAAGATCCTGCAGGTTTGTTTTATTCAAGTCCAGAAGAACTTGC
ATCAATGGGCGCGAAAATTAACATGGAACACAATGTGAAAAATATAGAT
AATGAGAATAAGGTCGTAGTAATTGAGAATTTAAAAACAGGCGAAACAT
TTGAAGAAAGCTATGATAAGTTGGTAATGACAACTGGATCATGGCCAATT
ATTCCTCCAATTGATGGAATCAATAGTGAAAATATTCTTTTGTGTAAAAA
CTATAACCAAGCAAATGAAATTATTAAAGAATCAAAAAATGCTAAAAAG
ATTGTCATTGTTGGTGGTGGCTATATTGGAATTGAATTAGTTGAGGCATTT
GCAGAATCTGGCAAGCAAGTGACGCTAGTTGATGGATTAGATCGTATTTT
AAACAAATATTTAGATGCTGAATTCACTTCTGTTTTAGAGCATGATTTACA
AGAAAGAGGCGTTACGCTAGCTTTAAACCAAACCGTCGAGAAATTTGTTG
CCAATGAATCAGGTGCTGTGACAGCTGTGAAAACACCAGTTGGAGAATA
TGAGGCTGATTTAGTTATTTTATGTGTTGGATTTAAACCAAATACTGATTT
GTTGAAGGATAAAGTAGAGATGTTGCCAAATGGTGCCATCGTAGTGGAT
GAATATATGAGAACAAGCGATGAAGCGATTTTTGCTGCTGGCGATAGTTG
CGCGGTTCATTATAATCCAACTGGAGGCTCTGCGTATATTCCGTTAGCTAC
AAATGCAGTTAGAATGGGAGCTTTAGTTGGGAAAAATATTGTTTCTCCAA
CAGTTAAATATCGTGGCACGCAAGCAACTTCTGGTTTATATTTATTTGGTT
TTAATATAGGTTCAACCGGATTGACTGAAAATAGCGCTCCTCATTTTGGC
GTAGAGGTTCGTTCAGTAGTTGTAGAAGATAATTATCGTCCAGAGTTTAT
GCCGACAACAGAGAAAGTAACGATGAAATTAGTTTATGAAGTAGGAACG
AATCGGATTGTTGGAGGTCAAATCATGTCAAAATATGATGTGACACAATC
TGCCAATACGTTATCTTTATGTGTTCAAAATAAAATGACGATTGAGGATT
TGGCTTATGTAGATTTCTTCTTCCAACCTCACTTTGATCGTCCTTGGAACT
ATTTAAATATTTTAGCGCAAGCAGCTGTTGAGCAAGAGCGTAAACTAGCA
AAATAA
SEQ ID No. 28:
MKVVVVGCTHAGTAAVKTILNEHPDASVSVYERNDNVSFLSCGIALYVGGV
VKDPAGLFYSSPEELASMGAKINMEHNVKNIDNENKVVVIENLKTGETFEE
SYDKLVMTTGSWPIIPPIDGINSENILLCKNYNQANEIIKESKNAKKIVIV
GGGYIGIELVEAFAESGKQVTLVDGLDRILNKYLDAEFTSVLEHDLQERGV
TLALNQTVEKFVANESGAVTAVKTPVGEYEADLVILCVGFKPNTDLLKDKV
EMLPNGAIVVDEYMRTSDEAIFAAGDSCAVHYNPTGGSAYIPLATNAVRMG
ALVGKNIVSPTVKYRGTQATSGLYLFGFNIGSTGLTENSAPHFGVEVRSVV
VEDNYRPEFMPTTEKVTMKLVYEVGTNRIVGGQIMSKYDVTQSANTLSLCV
QNKMTIEDLAYVDFFFQPHFDRPWNYLNILAQAAVEQERKLAK
SEQ ID No. 29:
ATGAGCAAAATTGTTATCGTGGGTGCAAATCATGCAGGCACCGCAGCAAT
TAATACCATTCTGGATAATTATGGCAGCGAAAATGAAGTGGTTGTGTTTG
ATCAGAATAGCAACATTAGCTTTCTGGGTTGTGGTATGGCACTGTGGATT
GGTAAACAAATTAGCGGTCCGCAGGGTCTGTTTTATGCAGATAAAGAAA
GCCTGGAAGCAAAAGGTGCCAAAATCTATATGGAAAGTCCGGTTACCGC
CATTGATTATGATGCAAAACGTGTTACCGCACTGGTTAATGGTCAAGAAC
ATGTTGAAAGCTACGAGAAACTGATTCTGGCAACCGGTAGCACCCCGATT
CTGCCTCCGATTAAAGGTGCAGCCATTAAAGAAGGTAGTCGCGATTTTGA
AGCAACCCTGAAAAATCTGCAGTTCGTGAAACTGTATCAGAATGCCGAA
GATGTGATTAACAAACTGCAGGATAAAAGCCAGAATCTGAATCGTATTGC
AGTTGTTGGTGCAGGTTATATTGGTGTTGAACTGGCAGAAGCATTTAAAC
GTCTGGGTAAAGAAGTGATTCTGATTGACGTTGTTGATACCTGTCTGGCA
GGTTATTATGATCAGGATCTGAGCGAAATGATGCGTCAGAATCTGGAAGA
TCATGGTATCGAACTGGCATTTGGTGAAACCGTTAAAGCAATTGAAGGTG
ATGGTAAAGTGGAACGTATTGTTACCGATAAAGCAAGCCATGATGTGGAT
ATGGTTATTCTGGCAGTTGGTTTTCGTCCGAATACAGCACTGGGTAATGC
AAAACTGAAAACCTTTCGTAATGGTGCCTTTCTGGTGGATAAAAAACAAG
AAACCAGCATCCCGGATGTTTATGCAATTGGTGATTGTGCAACCGTGTAT
GATAATGCCATTAACGACACCAACTATATTGCACTGGCAAGCAATGCACT
GCGTAGCGGTATTGTTGCAGGTCATAATGCAGCCGGTCATAAACTGGAAA
GTCTGGGTGTTCAGGGTAGCAATGGTATTTCAATTTTTGGCCTGAATATG
GTTAGCACCGGTCTGACCCAAGAAAAAGCCAAACGTTTTGGTTATAATCC
GGAAGTTACCGCCTTTACCGATTTTCAGAAAGCCAGCTTTATCGAGCATG
ATAACTATCCGGTTACGCTGAAAATTGTGTATGACAAAGATAGCCGTCTG
GTTCTGGGTGCACAGATGGCCAGCAAAGAAGATATGAGCATGGGTATTC
ACATGTTTAGCCTGGCCATTCAAGAGAAAGTTACCATTGAACGTCTGGCC
CTGCTGGATTATTTCTTTCTGCCGCATTTTAATCAGCCGTACAACTATATG
ACCAAAGCAGCACTGAAAGCCAAATAA
SEQ ID No. 30:
MSKIVIVGANHAGTAAINTILDNYGSENEVVVFDQNSNISFLGCGMALWIG
KQISGPQGLFYADKESLEAKGAKIYMESPVTAIDYDAKRVTALVNGQEHVE
SYEKLILATGSTPILPPIKGAAIKEGSRDFEATLKNLQFVKLYQNAEDVIN
KLQDKSQNLNRIAVVGAGYIGVELAEAFKRLGKEVILIDVVDTCLAGYYDQ
DLSEMMRQNLEDHGIELAFGETVKAIEGDGKVERIVTDKASHDVDMVILAV
GFRPNTALGNAKLKTFRNGAFLVDKKQETSIPDVYAIGDCATVYDNAINDT
NYIALASNALRSGIVAGHNAAGHKLESLGVQGSNGISIFGLNMVSTGLTQE
KAKRFGYNPEVTAFTDFQKASFIEHDNYPVTLKIVYDKDSRLVLGAQMASK
EDMSMGIHMFSLAIQEKVTIERLALLDYFFLPHENQPYNYMTKAALKAK
SEQ ID No. 31:
ATGAAAGTAGTAGTAGTAGGCTGTACACATGCAGGAACAGCGGCAGTTA
AGACGATTTTAAATGAACATCCAGATGCATCAGTATCAGTATATGAGCGT
AATGACAATGTCTCATTTCTATCTTGTGGGATTGCGTTGTATGTTGGTGGA
GTTGTGAAAGATCCTGCAGGTTTGTTTTATTCAAGTCCAGAAGAACTTGC
ATCAATGGGCGCGAAAATTAACATGGAACACAATGTGAAAAATATAGAT
AATGAGAATAAGGTCGTAGTAATTGAGAATTTAAAAACAGGCGAAACAT
TTGAAGAAAGCTATGATAAGTTGGTAATGACAACTGGATCATGGCCAATT
ATTCCTCCAATTGATGGAATCAATAGTGAAAATATTCTTTTGTGTAAAAA
CTATAACCAAGCAAATGAAATTATTAAAGAATCAAAAAATGCTAAAAAG
ATTGTCATTGTTGGTGGTGGCTATATTGCGATTGAATTAGTTGAGGCATTT
GCAGAATCTGGCAAGCAAGTGACGCTAGTTGCGCGTAGCGATCGTATTTT
ACGTAAATATTTAGATGCTGAATTCACTTCTGTTTTAGAGCATGATTTACA
AGAAAGAGGCGTTACGCTAGCTTTAAACCAAACCGTCGAGAAATTTGTTG
CCAATGAATCAGGTGCTGTGACAGCTGTGAAAACACCAGTTGGAGAATA
TGAGGCTGATTTAGTTATTTTATGTGTTGGATTTAAACCAAATACTGATTT
GTTGAAGGATAAAGTAGAGATGTTGCCAAATGGTGCCATCGTAGTGGAT
GAATATATGAGAACAAGCGATGAAGCGATTTTTGCTGCTGGCGATAGTTG
CGCGGTTCATTATAATCCAACTGGAGGCTCTGCGTATATTCCGTTAGCTAC
AAATGCAGTTAGAATGGGAGCTTTAGTTGGGAAAAATATTGTTTCTCCAA
CAGTTAAATATCGTGGCACGCAAGCAACTTCTGGTTTATATTTATTTGGTT
TTAATATAGGTTCAACCGGATTGACTGAAAATAGCGCTCCTCATTTTGGC
GTAGAGGTTCGTTCAGTAGTTGTAGAAGATAATTATCGTCCAGAGTTTAT
GCCGACAACAGAGAAAGTAACGATGAAATTAGTTTATGAAGTAGGAACG
AATCGGATTGTTGGAGGTCAAATCATGTCAAAATATGATGTGACACAATC
TGCCAATACGTTATCTTTATGTGTTCAAAATAAAATGACGATTGAGGATT
TGGCTTATGTAGATTTCTTCTTCCAACCTCACTTTGATCGTCCTTGGAACT
ATTTAAATATTTTAGCGCAAGCAGCTGTTGAGCAAGAGCGTAAACTAGCA
AAATAA
SEQ ID No. 32:
MKVVVVGCTHAGTAAVKTILNEHPDASVSVYERNDNVSFLSCGIALYVGGV
VKDPAGLFYSSPEELASMGAKINMEHNVKNIDNENKVVVIENLKTGETFEE
SYDKLVMTTGSWPIIPPIDGINSENILLCKNYNQANEIIKESKNAKKIVIV
GGGYIAIELVEAFAESGKQVTLVARSDRILRKYLDAEFTSVLEHDLQERGV
TLALNQTVEKFVANESGAVTAVKTPVGEYEADLVILCVGFKPNTDLLKDKV
EMLPNGAIVVDEYMRTSDEAIFAAGDSCAVHYNPTGGSAYIPLATNAVRMG
ALVGKNIVSPTVKYRGTQATSGLYLFGFNIGSTGLTENSAPHFGVEVRSVV
VEDNYRPEFMPTTEKVTMKLVYEVGTNRIVGGQIMSKYDVTQSANTLSLCV
QNKMTIEDLAYVDFFFQPHFDRPWNYLNILAQAAVEQERKLAK

Preferably, the H₂O-forming NAD(P)H oxidase that is used preferably comprises or consists of an amino acid sequence having an identity to SEQ ID No. 28, SEQ ID No. 30 or SEQ ID No. 32 of at least 80%, more preferably of 85%, more preferably of 90%, more preferably of 95%, more preferably of 98%, more preferably of 99%, in particular of 100%. Particularly preferably, the H₂O-forming NAD(P)H oxidase comprises or consists of the amino acid sequence SEQ ID No. 28, SEQ ID No. 30 or SEQ ID No. 32.

Alternatively, the H₂O-forming NAD(P)H oxidase preferably comprises an amino acid sequence encoded by a nucleic acid having an identity to SEQ ID No. 27, SEQ ID No. 29 or SEQ ID No. 31 of at least 80%, more preferably of 85%, more preferably of 90%, more preferably of 95%, more preferably of 98%, more preferably of 99%, in particular of 100%.

Particularly preferably, the nucleic acid encoding the H₂O-forming NAD(P)H oxidase comprises or consists of the nucleic acid sequence SEQ ID No. 27, SEQ ID No. 29 or SEQ ID No. 31.

A further aspect of the present invention relates to the use of a H₂O-forming NAD(P)H oxidase for cofactor regeneration (NAD(P)H to NAD(P)⁺) which comprises or consists of an amino acid sequence selected from the group consisting of:
i) an amino acid sequence having an identity to SEQ ID No. 28, SEQ ID No. 30 or SEQ ID No. 32 of at least 80%,
ii) an amino acid sequence encoded by a nucleic acid having an identity to SEQ ID No. 27, SEQ ID No. 29 or SEQ ID No. 31 of at least 80%, and
iii) an amino acid sequence encoded by a nucleic acid that binds under stringent conditions to a nucleic acid molecule having the nucleic acid sequence SEQ ID No. 27, SEQ ID No. 29 or SEQ ID No. 31.

### Materials

D-tagatose and D-talitol were purchased from TCI; D-psicose was purchased from TCI and Hunan Garden Naturals Inc. (China); D-fructose, NADPH tetrasodium salt, acetone, 2-propanol and methanol were purchased from PanReac AppliChem (ITW Reagents); zinc chloride, IPTG (isopropyl-β-D-thiogalactopyranoside) were purchased from Sigma-Aldrich; magnesium chloride hexahydrate, potassium dihydrogen phosphate, dipotassium hydrogen phosphate, NAD⁺, NADH disodium salt, NADP⁺ disodium salt and sodium dodecyl sulfate (SDS) were purchased from Carl Roth; sodium formate was purchased from Fluka; and triethanolamine (TEA) was purchased from Chem-Lab NV.

### Production of Enzymes & Preparation of Lysates

General Information on the Expression of Recombinant Enzymes in E. coli

For recombinant enzyme production in an Escherichia coli strain, the gene to be expressed was initially amplified in a PCR using, as a template, the genomic DNA or its equivalent that has been synthetically adapted to the codon usage of E. coli, together with specific oligonucleotides additionally carrying recognition sequences for restriction endonucleases, and was isolated from the reaction mixture. After nucleic acid digestion with the restriction enzymes SphI and HindIII, the gene fragment encoding the target enzyme was ligated into the SphI- and HindIII-cleaved backbone of the expression vector pQE70-Kan. The ligation product was transformed into chemically competent E. coli Top10F cells, and the resulting colonies were used for plasmid isolation and restriction analysis.

The result of the cloning step was verified by means of restriction enzyme digestion and DNA sequencing. The resulting construct carries the target gene under the IPTG-inducible T5 promoter.

For overexpression of the enzyme in E. coli, the resulting expression plasmid was transformed into the competent expression cells RB791. After 24 hours of incubation at 37° C., emerging colonies were inoculated in LB medium for expression assays.

On the following day, expression cultures with an optical density OD₅₅₀ of 0.02 were inoculated therewith and shaken at 37° C. until an OD₅₅₀ of 0.3 was reached. Subsequently, the temperature was lowered to 25° C., and the cultures were induced with 0.1 mM IPTG when an OD₅₅₀ of 0.5 was reached. After 22 hours, the cultures were harvested (separated from the medium in the form of a cell pellet by centrifugation) and analyzed with regard to the expression of the recombinant enzyme using SDS gel electrophoresis and activity determination (application in a use test or an optical-enzymatic assay).

### Preparation of Cell Lysates Using Sonifier Digestion

To prepare a cell suspension, the cell pellet produced according to the above-mentioned methods was weighed into a suitable container and mixed with buffer and lysozyme (final concentration of 0.5 mg/ml) (e.g., triethanolamine (TEA)-HCl) and was dissolved under stirring. The mass fraction of biomass is usually 20%, with the remainder being attributable to the buffer.

A Branson Sonifier 450 was used for the cell digestion. The suspension was treated three times with 15 ultrasonic pulses each (instrument settings: timer=15; duty cycle=50; output control=3-5).

The resulting homogenate was centrifuged for 10 minutes at 4° C. and 16,000 rpm (Eppendorf Centrifuge 5417R) in order to separate the insoluble cell fragments and obtain the lysate.

TABLE 1
Enzyme classes and donor organisms for the enzymes used in the examples.

| Enzyme type | | | |
|---|---|---|---|
| (EC class) | Catalyzed reaction | Donor organism | Literature/SEQ ID No. |
| D-psicose 3-epimerase | D-fructose ↔ D-psicose | Clostridium | (Mu et al., 2011; Chan et |
| (EC 5.1.3.30) | | cellulolyticum H10 | al., 2012) |
| glucose-isomerase | D-glucose ↔ D-fructose | Streptomyces murinus | Source: Sigma-Aldrich |
| | | | (G4166) |
| oxidoreductase I | D-psicose ↔ D-talitol | Caldilinea aerophila | SEQ ID No. 2 |
| oxidoreductase III | D-psicose ↔ D-talitol | Bacillus spizizenii | SEQ ID No. 6 |
| oxidoreductase IV | D-psicose ↔ D-talitol | Spathaspora | SEQ ID No. 8 |
| | | passalidarum | |
| oxidoreductase V | D-psicose ↔ D-talitol | Gluconobacter oxidans | SEQ ID No. 10 |
| oxidoreductase VI | D-psicose ↔ D-talitol | Millerozyma farinosa | SEQ ID No. 12 |
| oxidoreductase VII | D-psicose ↔ D-talitol | Debaryomyces fabryi | SEQ ID No. 14 |
| oxidoreductase VIII | D-psicose ↔ D-talitol | Torulaspora delbrueckii | SEQ ID No. 16 |
| oxidoreductase IX | D-psicose ↔ D-talitol | Sphaerochaeta | SEQ ID No. 18 |
| | | pleomorpha | |
| oxidoreductase X | D-psicose ↔ D-talitol | Leadbettera | SEQ ID No. 20 |
| | | azotonutricia | |
| oxidoreductase XI | D-psicose → D-talitol | Rasamsonia emersonii | SEQ ID No. 22 |
| oxidoreductase XII | D-psicose → D-talitol | Rasamsonia emersonii | SEQ ID No. 24 |
| oxidoreductase II | D-talitol → D-tagatose | Rasamsonia emersonii | SEQ ID No. 4 |
| alcohol dehydrogenase I | 2-propanol → acetone | (Geo-)Bacillus | (Sakoda & Imanaka, |
| (NAD) | | stearothermophilus | 1992) |
| (EC 1.1.1.1) | | NCA1503 | |
| alcohol dehydrogenase | 2-propanol ↔ acetone | Thermoanaerobium | (Lamed & Zeikus, 1980) |
| II (NADP) (EC 1.1.1.2) | | brockii | |
| NADH oxidase I | NADH → NAD⁺ | Carnobacterium | SEQ ID No. 28 |
| (EC 1.6.3.4) | | divergens | |
| NADH oxidase II | NADH → NAD⁺ | Streptococcus mutans | (Matsumoto et al., |
| (EC 1.6.3.4) | | | 1996); SEQ ID No. 30 |
| NAD(P)H oxidase (EC | NAD(P)H → NAD(P)⁺ | Carnobacterium | SEQ ID No. 32 |
| 1.6.3.2) | | divergens; mutant | |
| formate dehydrogenase | formate + NAD⁺ → | Starkeya novella | SEQ ID No. 26 |
| (EC 1.17.1.9) | CO₂ + NADH | | |

### Analytical Methods

### High Performance Liquid Chromatography

For the quantification of D-psicose, D-tagatose and D-fructose as well as of the intermediate D-talitol, using HPLC (high performance liquid chromatography) an Agilent HPLC 1260 Infinity II Series system was used. Detection was performed using a refractive index detector (RI detection). A Phenomenex Rezex RPM monosaccharide Pb+2 (80%) column with an appropriate precolumn was used for the measurement, and isocratic elution was done with ultrapure water.

### Determination of Enzyme Activities (Optical Enzymatic Assay)

Enzyme activities in the lysates were determined by means of a Shimadzu UV-1900 spectrophotometer. For this purpose, the formation and, respectively, the consumption of NAD(P)H were monitored at a wavelength of 340 nm via the change in absorption. The measurements were performed with 0.2 mM cofactor (NAD(P)⁺ or NAD(P)H). For this purpose, 20 μl of a 10 mM stock solution of the cofactor was placed in a flask (Greiner bio-one semi-micro polystyrene flask), and the desired pH value was adjusted with 100 mM TEA-HCl buffer (870 μl). 10 μl of lysate (diluted or undiluted) and 100 μl of substrate solution were added into the flask, and the measurement was started immediately afterwards. The measurements were performed at 25° C. by default. Via the extinction coefficient of NAD(P)H at 340 nm (ε=6220 L mol⁻¹ cm⁻¹), the enzyme activity of the lysate can be determined in U/ml (based on the volume of the lysate) or in U/g (based on the biomass used for production). In this context, 1 U represents 1 μmol of substrate turnover per minute (1 U=1 μmol/min=1.67·10⁻⁸ cat).

Preferred variants of the process according to the invention are described in more detail by way of the following examples. The lysates used in these examples were produced according to the processes described above.

### Example 1

Production of D-Talitol from D-Fructose—Cofactor Regeneration with Alcohol Dehydrogenase

The reaction was performed in a Labfors 5 benchtop bioreactor (Infors AG). A glass reactor (volume of 3.4 l) with a stirrer attached and a pH electrode was used as a vessel. pH control was achieved by metered addition of 1M NaOH or 1M H₂SO₄.

At the beginning, 50 ml of a D-fructose solution (500 g/l), 238.7 ml deionized water and 89 ml of a 200 mM TEA-HCl buffer (pH 7.5) were placed in the reactor and brought to 35° C. under stirring.

To start the reaction, 25 ml of D-psicose 3-epimerase lysate was added. Subsequently, 35 ml of oxidoreductase I lysate, 8 kU of alcohol dehydrogenase I lysate, 10 ml of a 10 mM NAD⁺ solution and 40 ml of 2-propanol were introduced.

During operation, samples were continuously taken from the reactor solution and analyzed as follows: 100 μl of the reactor solution was mixed with 200 μl methanol and incubated in an Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

After 24 hours and a runtime of 42 hours, 15 ml of 2-propanol was added in each case.

After 67 hours, 98.0% of the D-fructose was converted to D-talitol (detected concentration: 45.1 g/l).

Afterwards, the reactor contents were heated to 70° C. while stirring for 30 minutes. The hot mixture was filtered through a pleated filter (Schleicher & Schuell 595½, 185 mm). The filtrate was stirred with activated carbon at 50° C. for 30 minutes, whereupon the activated carbon was filtered off (Schleicher & Schuell 595½ pleated filter, 185 mm). The colourless solution was treated with a mixed-bed ion exchange resin (Amberlite MB20) at 50° C. for 30 minutes, which was then removed by filtration (Schleicher & Schuell 595½ pleated filter, 185 mm). The solution was concentrated on a rotary evaporator (60° C., 150-60 mbar) up to a concentration of approximately 600 g/l D-talitol. The resulting syrup was incubated overnight in a refrigerator at 4° C. Since crystal formation could not be observed, a seed crystal was added, and the syrup was again stored overnight in the refrigerator at 4° C. The syrup had the same volume of IPA added to it, and it was stored in a freezer at −20° C. to induce crystallization. Upon application of a vacuum, the colourless crystals were filtered off through a glass frit (P4, 10-16 μm) and dried in a vacuum drying oven at 50° C. for 24 hours. HPLC analysis of the crystals (50% yield) revealed that the product was D-talitol (94% purity).

### Example 2

Production of D-Talitol from D-Fructose—Cofactor Regeneration with Formate Dehydrogenase

The reaction was performed in a Labfors 5 benchtop bioreactor (Infors AG). A glass reactor (volume of 3.4 l) with a stirrer attached and a pH electrode was used as a vessel. pH control was achieved by metered addition of 1M NaOH or 1M H₂SO₄.

At the beginning, 17.5 g of D-fructose, 125 ml deionized water and 4.2 ml of a 500 mM TEA-HCl buffer (pH 7.5) were placed in the reactor and brought to 35° C. under stirring.

To start the reaction, 17.5 ml of D-psicose 3-epimerase lysate was added. Subsequently, 4.7 kU of oxidoreductase I lysate, 5.5 kU of formate dehydrogenase lysate, 3.5 ml of a 10 mM NAD⁺ solution and 45.5 ml of a 5 M sodium formate solution were introduced. Air was blown into the reaction mixture to remove the CO₂ produced by the formate dehydrogenase.

During operation, samples were continuously taken from the reactor solution and analyzed as follows: 100 μl of the reactor solution was mixed with 200 μl methanol and incubated in an Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

After 26 hours, 99% of the D-fructose (50 g/l) was converted to D-talitol (detected concentration: 34.4 g/l).

Afterwards, the reactor contents were heated to 70° C. with stirring for 30 minutes. The hot mixture was filtered through a pleated filter (Schleicher & Schuell 595½, 185 mm). The filtrate was stirred with activated carbon at 50° C. for 30 minutes, whereupon the activated carbon was filtered off (Schleicher & Schuell 595½ pleated filter, 185 mm). The colourless solution was treated with a mixed-bed ion exchange resin (Amberlite MB20) at 50° C. for 30 minutes, which was then removed by filtration (Schleicher & Schuell 595½ pleated filter, 185 mm). The solution was concentrated to a concentration of approximately 600 g/l D-talitol on a rotary evaporator (60° C., 150-60 mbar). The resulting syrup was incubated overnight in a refrigerator at 4° C. Since crystal formation could not be observed, a seed crystal was added, and the syrup was again stored overnight in the refrigerator at 4° C. The syrup had the same volume of IPA added to it, and it was stored in a freezer at −20° C. to induce crystallization. Upon application of a vacuum, the colourless crystals were filtered off through a glass frit (P4, 10-16 μm) and dried in a vacuum drying oven at 50° C. for 24 hours. HPLC analysis of the crystals (50% yield) revealed that the product was D-talitol (94% purity).

### Example 3

Oxidation of D-Talitol to D-Tagatose (Cofactor Regeneration with NAD(P)H Oxidase)

The following components were mixed in a 2 ml glass vial: 147.2 μl deionized water, 125 μl of a 400 mM TEA-HCl buffer (pH 8.5), 150 μl of a D-talitol solution (168 g/l), 35 μl of oxidoreductase II lysate, 10 U of NAD(P)H oxidase lysate, and 5 μl of a 5 mM NADP⁺ solution. The batch was incubated in an Eppendorf Thermomixer with continuous shaking (24° C., 800 rpm) for 20 hours.

For analysis, 100 μl of the batch was mixed with 200 μl methanol and incubated in the Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

In this way, 81.7% of the D-talitol was converted to D-tagatose (detected concentration: 47.7 g/l).

### Example 4

Oxidation of D-Talitol to D-Tagatose (Cofactor Regeneration with Alcohol Dehydrogenase)

The reaction was performed in a Labfors 5 benchtop bioreactor (Infors AG). A glass reactor (volume of 3.4 l) with a stirrer attached and a pH electrode was used as a vessel. pH control was achieved by metered addition of 1M NaOH or 1M H₂SO₄.

At the beginning, 43.8 ml of a D-talitol solution (571 g/l), 379.5 ml deionized water and 43.4 ml of a 500 mM TEA-HCl buffer (pH 8) were placed in the reactor and brought to 30° C. under stirring.

To start the reaction, 25 ml of oxidoreductase II lysate, 12 kU of alcohol dehydrogenase II lysate and 25 ml acetone were introduced. In addition, overpressure was applied to minimize evaporation losses.

During operation, samples were continuously taken from the reactor solution and analyzed as follows: 100 μl of the reactor solution was mixed with 200 μl methanol and incubated in an Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

After 14 hours, 25 ml of oxidoreductase II lysate, 12 kU of alcohol dehydrogenase II lysate and 5 ml of a 10 mM NADP⁺ solution were introduced.

After 34 hours, 43% of the D-talitol was converted to D-tagatose (detected concentration: 18.1 g/l).

### Example 5

Production of D-Tagatose from D-Fructose (One-Pot Process)

The reaction was performed in a Labfors 5 benchtop bioreactor (Infors AG). A glass reactor (volume of 3.4 l) with a stirrer attached, a pH electrode and an O₂ sensor was used as a vessel. pH control was achieved by metered addition of 1M NaOH or 1M H₂SO₄.

At the beginning, 50 ml of a D-fructose solution (500 g/l), 238.8 ml deionized water and 89 ml of a 200 mM TEA-HCl buffer (pH 7.5) were placed in the reactor and brought to 35° C. under stirring.

To start the reaction, 25 ml of D-psicose 3-epimerase lysate was added. Subsequently, 35 ml of oxidoreductase I lysate, 8 kU of alcohol dehydrogenase I lysate, 10 ml of a 10 mM NAD⁺ solution and 40 ml of 2-propanol were introduced.

During operation, samples were continuously taken from the reactor solution and analyzed as follows: 100 μl of the reactor solution was mixed with 200 μl methanol and incubated in an Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

After a runtime of 24 hours, 15 ml of 2-propanol was added.

After 47 hours, 94.0% of the D-fructose was converted to D-talitol (detected concentration: 41.3 g/l).

After cooling the reactor contents to 24° C., 15 ml of oxidoreductase II lysate, 5 kU of NAD(P)H oxidase lysate and 10 ml of a 5 mM NADP⁺ solution were added. Additionally, the pH value of the reactor solution was increased to 8.

After a total runtime of 97 hours, the reactor contents were heated to 70° C., the pH value was adjusted to 4, and stirred for 30 minutes at 70° C. The precipitate was filtered off through a glass frit (P3).

29.9 g/l of D-tagatose was detected in the filtrate.

The filtrate was concentrated to a D-tagatose concentration of 257 g/l on a rotary evaporator.

### Example 6

### Oxidation of D-Talitol to D-Psicose

The following components were mixed in a 2 ml glass vial: 30 μl deionized water, 250 μl of a 200 mM TEA-HCl buffer (pH 8), 125 μl of a D-talitol solution (200 g/l), 50 μl of oxidoreductase I lysate, 40 μl of NADH oxidase lysate, and 5 μl of a 10 mM NAD⁺ solution. The batch was incubated in an Eppendorf Thermomixer under continuous shaking (30° C., 800 rpm) for 20 hours.

For analysis, 100 μl of the batch was mixed with 200 μl methanol and incubated in the Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

In this way, 75.3% of the D-talitol was converted to D-psicose (detected concentration: 39.3 g/l).

### Example 7

Production of D-Psicose from D-Fructose (One-Pot Method)

The following components were mixed in a 2 ml glass vial: 73.8 μl deionized water, 250 μl of a 200 mM TEA-HCl buffer (pH 7.5), 50 μl of a D-fructose solution (500 g/l), and 25 μl of D-psicose 3-epimerase lysate. Subsequently, 35 μl of oxidoreductase I lysate, 8 U of alcohol dehydrogenase I lysate, 5 μl of a 10 mM NAD⁺ solution and 50 μl of 2-propanol were added to the batch. The batch was incubated with continuous shaking (35° C., 800 rpm) for a total of 20 hours.

The batch was then heated to 70° C. for 60 minutes. After cooling, 25 μl of oxidoreductase I lysate, 10 U of NADH oxidase lysate, 5 μl of a 5 mM NAD⁺ solution and 100 ml deionized water were added. The batch was incubated in an Eppendorf Thermomixer under continuous shaking (24° C., 800 rpm) for another 20 hours.

For analysis, 100 μl of the batch was mixed with 200 μl methanol and incubated in the Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

In this way, 76.9% of the D-fructose (50 g/l) was converted to D-psicose (detected concentration: 39.9 g/l).

### Example 8

### Reduction of D-Psicose to D-Talitol

The reaction was performed in a Labfors 5 benchtop bioreactor (Infors AG). A glass reactor (volume of 3.4 l) with a stirrer attached and a pH electrode was used as a vessel. pH control was achieved by metered addition of 1M NaOH or 1M H₂SO₄.

At the beginning, 98.8 ml of a D-psicose solution (508 g/l), 270.9 ml deionized water and 41.2 ml of a 500 mM TEA-HCl buffer (pH 7.5) were placed in the reactor and brought to 35° C. under stirring.

To start the reaction, 35 ml of oxidoreductase VII lysate, 12 kU of alcohol dehydrogenase II lysate, 5 ml of a 10 mM NADP⁺ solution and 40 ml of 2-propanol were introduced.

During operation, samples were continuously taken from the reactor solution and analyzed as follows: 100 μl of the reactor solution was mixed with 200 μl methanol and incubated in an Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

After a runtime of 24 hours and 48 hours, 20 ml of 2-propanol was added in each case.

After 25 hours, 94% of the D-psicose (100 g/l) could be reduced to D-talitol—after 52 hours, the reduction was complete (see FIG. 2).

To separate the enzymes, the reactor contents were heated to 70° C., the pH value was adjusted to 4, and stirring took place at 70° C. for 30 minutes. The precipitate was filtered off through a glass frit (P3).

The D-talitol concentration overtime is illustrated in FIG. 2. A comparison with the time curves in the publication (FIG. 6) by Sasahara et al. (1998) shows that the reduction of D-psicose (10%=100 g/l) to D-talitol can be accomplished with an in vitro system within approximately 1/10 of the time.

### Example 9

Production of D-Talitol from D-Glucose (One-Pot Process)

In this example, D-fructose is generated in situ from D-glucose using glucose isomerase and is reacted further to D-talitol.

The following components were mixed in a 2 ml glass vial: 95.2 μl deionized water, 100 μl of a 500 mM TEA-HCl buffer (pH 8), 100 μl of a D-glucose solution (500 g/l), 50 μl of a 5 mM MgSO₄ solution, and 15 mg of glucose isomerase (Streptomyces murinus; Sigma-Aldrich G4166).

Subsequently, 25 μl of D-psicose 3-epimerase lysate was first added, followed by 35 μl of oxidoreductase IX lysate, 12 U of alcohol dehydrogenase I lysate, 5 μl of a 10 mM NAD⁺ solution and 50 μl of 2-propanol. The batch was incubated under continuous shaking (40° C., 800 rpm) for a total of 20 hours.

For analysis, 100 μl of the batch was mixed with 200 μl methanol and incubated in the Eppendorf Thermomixer at 60° C. and 1200 rpm for 15 minutes. The sample was briefly centrifuged in a centrifuge, mixed with 700 μl deionized water, vortexed, and subsequently centrifuged at max. g for 5 minutes. 200 μl of the supernatant was transferred into an HPLC vial with an insert and surveyed by HPLC (RI detection).

After 20 hours, 100 g/l of D-glucose could be converted to 60 g/l of D-talitol.

### LITERATURE

Reed, R. H., Wright, P. J., Chudek, J. A., & Hunter, G. (1995). Turnover of hexitols in the marine macroalga Himanthalia elongata (Phaeophyta, Fucales). European Journal of Phycology, 30(3), 169-177. https://doi.org/10.1080/09670269500650951
Li, Z., Gao, Y., Nakanishi, H., Gao, X., & Cai, L. (2013). Biosynthesis of rare hexoses using microorganisms and related enzymes. Beilstein Journal of Organic Chemistry, 9, 2434-2445. https://doi.org/10.3762/bjoc.9.281
Izumori, K. (2006). Izumoring: a strategy for bioproduction of all hexoses. Journal of Biotechnology, 124(4), 717-722. https://doi.org/10.1016/j.jbiotec.2006.04.016 Hirst, E. L., Hough, L., & Jones, J. K. N. (1949). Composition of the Gum of Sterculia setigera: Occurrence of D-Tagatose in Nature. Nature, 163, 177. https://doi.org/10.1038/163177b0
Lindberg, B. (1955). Studies on the Chemistry of Lichens. VIII. Investigation of a Dermatocarpon and Some Roccella Species. Acta Chemica Scandinavica, 9, 917-919. https://doi.org/10.3891/acta.chem.scand.09-0917
Adachi, S. (1958). Formation of Lactulose and Tagatose from Lactose in strongly heated Milk. Nature, 181, 840-841 (1958). https://doi.org/10.1038/181840a0
Hough, L., & Stacey, B. E. (1963). The occurrence of D-ribohexulose in Itea ilicifolia, Itea virginica, and Itea yunnanensis. Phytochemistry, 2(4), 315-320. https://doi.org/10.1016/S0031-9422(00)84854-2
Oshima, H., Kimura, I., & Izumori, K. (2006). Psicose Contents in Various Food Products and its Origin. Food Science and Technology Research, 12(2), 137-143. https://doi.org/10.3136/fstr.12.137
Jiang, S., Xiao, W., Zhu, X., Yang, P., Zheng, Z., Lu, S., Jiang, S., Zhang, G., & Liu, J. (2020). Review on D-Allulose: In vivo Metabolism, Catalytic Mechanism, Engineering Strain Construction, Bio-Production Technology. Frontiers in Bioengineering and Biotechnology, 8, 26. https://doi.org/10.3389/fbioe.2020.00026
Oh, D.-K. (2007). Tagatose: properties, applications, and biotechnological processes. Applied Microbiology and Biotechnology, 76, 1-8. https://doi.org/10.1007/s00253-007-0981-1
Roy, S., Chikkerur, J., Roy, S. C., Dhali, A., Kolte, A. P., Sridhar, M., & Samanta, A. K. (2018). Tagatose as a Potential Nutraceutical: Production, Properties, Biological Roles, and Applications. Journal of Food Science, 83(11), 2699-2709. https://doi.org/10.1111/1750-3841.14358
Levin, G. V. (2002). Tagatose, the New GRAS Sweetener and Health Product. Journal of Medical Food, 5(1), 23-36. https://doi.org/10.1089/109662002753723197
Ahmed, A., Khan, T. A., Ramdath, D. D., Kendall, C. W. C., & Sievenpiper, J. L. (2022). Rare sugars and their health effects in humans: a systematic review and narrative synthesis of the evidence from human trials, Nutrition Reviews, 80(2), 255-270. https://doi.org/10.1093/nutrit/nuab012
Mochizuki, S., Fukumoto, T., Ohara, T., Ohtani, K., Yoshihara, A., Shigematsu, Y., Tanaka, K., Ebihara, K., Tajima, S., Gomi, K., Ichimura, K., Izumori. K., & Akimitsu, K. (2020). The rare sugar D-tagatose protects plants from downy mildews and is a safe fungicidal agrochemical. Communications Biology, 3, 423. https://doi.org/10.1038/s42003-020-01133-7
Chahed, A., Nesler, A., Aziz, A., Barka, E. A., Pertot, I., & Perazzolli, M. (2021). A review of knowledge on the mechanisms of action of the rare sugar D-tagatose against phytopathogenic oomycetes. Plant Pathology, 70(9), 1979-1986. https://doi.org/10.1111/ppa.13440
Zhang, W., Yu, S., Zhang, T., Jiang, B., & Mu, W. (2016). Recent advances in D-allulose: Physiological functionalities, applications, and biological production. Trends in Food Science and Technology, 54, 127-137. https://doi.org/10.1016/j.tifs.2016.06.004
Chen, Z., Gao, X.-D., & Li, Z. (2022). Recent Advances Regarding the Physiological Functions and Biosynthesis of D-Allulose. Frontiers in Microbiology, 13, 881037. https://doi.org/10.3389/fmicb.2022.881037
Heath, E. C., Horecker, B. L., Smyrniotis, P. Z., & Takagi, Y. (1958). Pentose fermentation by Lactobacillus plantarum. II. L-arabinose isomerase. Journal of Biological Chemistry, 231(2), 1031-1037 (1958). https://doi.org/10.1016/S0021-9258(18)70464-X
Dai, Y., Zhang, J., Zhang, T., Chen, J., Hassanin, H. A. M., & Jiang, B. (2020). Characteristics of a fructose 6-phosphate 4-epimerase from Caldilinea aerophila DSM 14535 and its application for biosynthesis of tagatose. Enzyme and Microbial Technology, 139, 109594. https://doi.org/10.1016/j.enzmictec.2020.109594
Lee, S.-H., Hong, S.-H., Kim, K.-R., & Oh, D.-K. (2017). High-yield production of pure tagatose from fructose by a three-step enzymatic cascade reaction. Biotechnology Letters, 39, 1141-1148. https://doi.org/10.1007/s10529-017-2340-3
Shaw, D. R. D. (1956). Polyol dehydrogenases. 3. Galactitol dehydrogenase and D-iditol dehydrogenase. Biochemical Journal, 64(3), 394-405. https://doi.org/10.1042/bj0640394
Shin, K.-C., Lee, T.-E., Seo, M.-J., Kim, D. W., Kang, L.-W. & Oh, D.-K. (2020). Development of Tagaturonate 3-Epimerase into Tagatose 4-Epimerase with a Biocatalytic Route from Fructose to Tagatose. ACS Catalysis, 10(20), 12212-12222. https://doi.org/10.1021/acscatal.002922
Jeon, E. J., Lee, Y.-M., Choi, E. J., Kim, S.-B., & Jeong, K. J. (2023). Production of Tagatose by Whole-cell Bioconversion from Fructose Using Corynebacterium glutamicum. Biotechnology and Bioprocess Engineering. https://doi.org/10.1007/si2257-022-0304-5
Izumori, K., Khan, A. R., Okaya, H., & Tsumura, T. (1993). A New Enzyme, D-Ketohexose 3-Epimerase, from Pseudomonas sp. ST-24. Bioscience, Biotechnology, and Biochemistry, 57(6), 1037-1039. https://doi.org/10.1271/bbb.57.1037
Van Duc Long, N., Le, T.-H., Kim, J.-I., Lee, J. W. and Koo, Y.-M. (2009). Separation of D-psicose and D-fructose using simulated moving bed chromatography. Journal of Separation Science, 32(11), 1987-1995. https://doi.org/10.1002/jssc.200800753
Li, Y., Shi, T., Han, P., & You, C. (2021). Thermodynamics-Driven Production of Value-Added D-Allulose from Inexpensive Starch by an In Vitro Enzymatic Synthetic Biosystem. ACS Catalysis, 11(9), 5088-5099. https://doi.org/10.1021/acscatal.0c05718
Wang, W., Yang, J., Sun, Y., Li, Z., & You, C. (2020). Artificial ATP-Free in Vitro Synthetic Enzymatic Biosystems Facilitate Aldolase-Mediated C—C Bond Formation for Biomanufacturing. ACS Catalysis, 10(2), 1264-1271. https://doi.org/10.1021/acscatal.9b04696
Li, Z., Li, F., Cai, L., Chen, Z., Qin, L., & Gao, X.-D. (2020). One-Pot Multienzyme Synthesis of Rare Ketoses from Glycerol. Journal of Agricultural and Food Chemistry, 68(5), 1347-1353. https://doi.org/10.1021/acs.jafc.9b06748
Xiao, Q., Niu, J., Liu, H., Liu, Y., & Zhou, X. (2019). High Conversion of D-Fructose into D-Allulose by Enzymes Coupling with an ATP Regeneration System. Molecular Biotechnology, 61, 432-441. https://doi.org/10.1007/si2033-019-00174-6
Takeshita, K., Ishida, Y., Takada, G., & Izumori, K. (2000). Direct Production of Allitol from D-Fructose by a Coupling Reaction Using D-Tagatose 3-Epimerase, Ribitol Dehydrogenase and Formate Dehydrogenase. Journal of Bioscience and Bioengineering, 90(5), 545-548. https://doi.org/10.1016/S1389-1723(01)80038-4
Wang, L., Chen, K., Zheng, P., Huo, X., Liao, F., Zhu, L., Hu, M., & Tao, Y. (2023). Enhanced production of D-psicose from D-fructose by a redox-driven multi-enzyme cascade system. Enzyme and Microbial Technology, 163, 110172. https://doi.org/10.1016/j.enzmictec.2022.110172
Izumori, K. (2002). Bioproduction strategies for rare hexose sugars. Naturwissenschaften, 89, 120-124. https://doi.org/10.1007/s00114-002-0297-z
Boverio, A., van Beek, H. L., Savino, S., Ranoux, A., Huijgen, W. J. J., Raaijmakers, H. W. C., Fraaije, M. W., & Loncar, N. (2023). Biochemical and Structural Characterization of a Uronic Acid Oxidase from Citrus sinensis. ChemCatChem, 15(21), e202300847. https://doi.org/10.1002/cctc.202300847
Vastano, M., Pellis, A., Botelho Machado, C., Simister, R., McQueen-Mason, S. J., Farmer, T. J., & Gomez, L. D. (2019). Sustainable Galactarate-Based Polymers: Multi-Enzymatic Production of Pectin-Derived Polyesters. Macromolecular Rapid Communications, 40(22), 1900361. https://doi.org/10.1002/marc.201900361
Muniruzzaman, S., Kobayashi, H., & Izumori, K. (1994). Production of D-talitol from D-tagatose by Aureobasidium pullulans strain 113B. Journal of Fermentation and Bioengineering, 78(5), 346-350. https://doi.org/10.1016/0922-338X(94)90278-X
Wichelecki, D. J., Vetting, M. W., Chou, L., Al-Obaidi, N., Bouvier, J. T., Almo, S. C., & Gerlt, J. A. (2015). ATP-binding Cassette (ABC) Transport System Solute-binding Protein-guided Identification of Novel D-Altritol and Galactitol Catabolic Pathways in Agrobacterium tumefaciens C58. The Journal of Biological Chemistry, 290(48), 28963-28976. https://doi.org/10.1074/jbc.M115.686857
Izumori, K., Yamakita, M., Tsumura, T., & Kobayashi, H. (1990). Production of D-psicose from D-talitol, D-tagatose or D-galactitol by Alcaligenes sp. 701B. Journal of Fermentation and Bioengineering, 70(1), 26-29. https://doi.org/10.1016/0922-338X(90)90025-R
Yoshihara, K., Shinohara, Y., Hirotsu, T., & Izumori, K. (2006). Bioconversion of D-psicose to D-tagatose and D-talitol by Mucoraceae fungi. Journal of Bioscience & Bioengineering, 101(3), 219-222. https://doi.org/10.1263/jbb.101.219
Sasahara, H., Mine, M., & Izumori, K. (1998). Production of D-talitol from D-psicose by Candida famata R28. Journal of Fermentation and Bioengineering, 85(1), 84-88. https://doi.org/10.1016/S0922-338X(97)80359-5
Xu, J., Zhou, H., Yu, H., Deng, T., Wang, Z., Zhang, H., Wu, J., & Yang, L. (2021). Computational design of highly stable and soluble alcohol dehydrogenase for NADPH regeneration. Bioresources and Bioprocessing, 8, 12. https://doi.org/10.1186/s40643-021-00362-w
Al-Rabiah, A. A., Boz, I., Akhmedov, V. M., Mostafa, M. M. M., & Bagabas, A. A. (2022). Highly Selective Gas-Phase Catalytic Hydrogenation of Acetone to Isopropyl Alcohol. Catalysts, 12(10), 1251. https://doi.org/10.3390/cata112101251
Lin, B., & Tao, Y. (2017). Whole-cell biocatalysts by design. Microbial Cell Factories, 16, 106. https://doi.org/10.1186/s12934-017-0724-7
Altschul, S. F., Gish, W., Miller, W., Myers, E. W., & Lipman, D. J. (1990). Basic local alignment search tool. Journal of Molecular Biology, 215(3), 403-410. https://doi.org/10.1016/S0022-2836(05)80360-2
Henikoff, S., & Henikoff, J. G. (1992). Amino acid substitution matrices from protein blocks. Proceedings of the National Academy of Sciences of the United States of America, 89(22), 10915-10919. https://doi.org/10.1073/pnas.89.22.10915
Sambrook, J., Fritsch, E. R., & Maniatis, T. (1989). Molecular Cloning: A Laboratory Manual (2nd ed.). Cold Spring Harbor, NY: Cold Spring Harbor Laboratory Press.
Mu, W., Chu, F., Xing, Q., Yu, S., Zhou, L., & Jiang, B. (2011). Cloning, Expression, and Characterization of a D-Psicose 3-Epimerase from Clostridium cellulolyticum H10. Journal of Agricultural and Food Chemistry, 59(14), 7785-7792. https://doi.org/10.1021/jf201356q
Chan, H.-C., Zhu, Y., Hu, Y., Ko, T.-P., Huang, C.-H., Ren, F., Chen, C.-C., Ma, Y., Guo, R.-T., & Sun, Y. (2012). Crystal structures of D-psicose 3-epimerase from Clostridium cellulolyticum H10 and its complex with ketohexose sugars. Protein & Cell, 3(2), 123-131. https://doi.org/10.1007/si3238-012-2026-5
Sakoda, H., & Imanaka, T. (1992). Cloning and sequencing of the gene coding for alcohol dehydrogenase of Bacillus stearothermophilus and rational shift of the optimum pH. Journal of Bacteriology, 174(4), 1397-1402. https://doi.org/10.1128/jb.174.4.1397-1402.1992
Lamed, R., & Zeikus, J. G. (1980). Ethanol production by thermophilic bacteria: relationship between fermentation product yields of and catabolic enzyme activities in Clostridium thermocellum and Thermoanaerobium brockii. Journal of Bacteriology, 144(2), 569-578. https://doi.org/10.1128/jb.144.2.569-578.1980
Matsumoto, J., Higuchi, M., Shimada, M., Yamamoto, Y., & Kamio, Y. (1996). Molecular Cloning and Sequence Analysis of the Gene Encoding the H₂O-forming NADH Oxidase from Streptococcus mutans. Bioscience, Biotechnology, and Biochemistry, 60(1), 39-43. https://doi.org/10.1271/bbb.60.39

1. A method for the production of an aqueous solution containing D-talitol in that D-psicose is formed in vitro from D-fructose, which exists in a dissolved state in an aqueous solution, by treating it with an epimerase, whereupon the D-psicose is reduced in vitro to D-talitol by treating it with an NAD(P)H-dependent oxidoreductase, and the epimerase and the oxidoreductase are separated or deactivated.

2. A method for the production of D-talitol, wherein the aqueous solution produced according to claim 1 is concentrated and the D-talitol is crystallized out.

3. A method according to claim 1, wherein the oxidized cofactor NAD(P)⁺ formed by the reduction is reduced by means of an alcohol dehydrogenase, and a secondary alcohol, thereby forming a ketone.

4. A method according to claim 3, wherein the secondary alcohol is 2-propanol.

5. A method according to claim 1, wherein the oxidized cofactor NAD(P)⁺ formed by the reduction is reduced by means of a formate dehydrogenase and formate, thereby forming carbon dioxide.

6. A method according to claim 1, wherein the NAD(P)H-dependent oxidoreductase for the reduction of D-psicose to D-talitol comprises an amino acid sequence selected from the group consisting of:
i) an amino acid sequence having an identity to SEQ ID No. 2, SEQ ID No. 6, SEQ ID No. 8, SEQ ID No. 10, SEQ ID No. 12, SEQ ID No. 14, SEQ ID No. 16, SEQ ID No. 18, SEQ ID No. 20, SEQ ID No. 22, or SEQ ID No. 24 of at least 80%,
ii) an amino acid sequence encoded by a nucleic acid having an identity to SEQ ID No. 1, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, or SEQ ID No. 23 of at least 80%, and
iii) an amino acid sequence encoded by a nucleic acid that binds under stringent conditions to a nucleic acid molecule having the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17, SEQ ID No. 19, SEQ ID No. 21, or SEQ ID No. 23.

7. A method for the production of D-talitol or D-psicose, comprising the step of treating D-psicose or D-talitol with an oxidoreductase comprising an amino acid sequence selected from the group consisting of:
i) an amino acid sequence having an identity to SEQ ID No. 2, SEQ ID No. 6, SEQ ID No. 8, SEQ ID No. 10, SEQ ID No. 12, SEQ ID No. 14, SEQ ID No. 16, SEQ ID No. 18, or SEQ ID No. 20 of at least 80%,
ii) an amino acid sequence encoded by a nucleic acid having an identity to SEQ ID No. 1, SEQ ID No. 5, SEQ ID No. 7, SEQ ID No. 9, SEQ ID No. 11, SEQ ID No. 13, SEQ ID No. 15, SEQ ID No. 17, or SEQ ID No. 19 of at least 80%, and
iii) an amino acid sequence encoded by a nucleic acid that binds under stringent conditions to a nucleic acid molecule having the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17, or SEQ ID No. 19.

8. A method for the production of D-talitol, comprising the step of treating D-psicose with an oxidoreductase comprising an amino acid sequence selected from the group consisting of:
i) an amino acid sequence having an identity to SEQ ID No. 22 or SEQ ID No. 24 of at least 80%,
ii) an amino acid sequence encoded by a nucleic acid having an identity to SEQ ID No. 21 or SEQ ID No. 23 of at least 80%, and
iii) an amino acid sequence encoded by a nucleic acid that binds under stringent conditions to a nucleic acid molecule having the nucleic acid sequence SEQ ID No. 21 or SEQ ID No. 23.

9. (canceled)

10. (canceled)

11. A method for the production of an aqueous solution of D-tagatose or D-psicose, wherein the aqueous solution containing D-talitol, which has been prepared by the process according to claim 1, is treated in vitro with an NAD(P)⁺-dependent oxidoreductase, thereby forming the reduced cofactor NAD(P)H, in order to oxidize D-talitol to D-tagatose or, respectively, D-talitol to D-psicose, whereupon the oxidoreductase is separated.

12. A method according to claim 11, wherein the reduced cofactor NAD(P)H formed by the oxidation is oxidized to NAD(P)⁺ by means of an oxidase and oxygen.

13. A method according to claim 11, wherein the reduced cofactor NAD(P)H formed by the oxidation is oxidized to NAD(P)⁺ by means of an alcohol dehydrogenase and a ketone.

14. A method according to claim 13, wherein the ketone is acetone.

15. A method according to claim 12, wherein the oxidase for the oxidation of cofactor NAD(P)H to NAD(P)⁺ comprises an amino acid sequence selected from the group consisting of:
i) an amino acid sequence having an identity to SEQ ID No. 28, SEQ ID No. 30, or SEQ ID No. 32 of at least 80%,
ii) an amino acid sequence encoded by a nucleic acid having an identity to SEQ ID No. 27, SEQ ID No. 29, or SEQ ID No. 31 of at least 80%, and
iii) an amino acid sequence encoded by a nucleic acid that binds under stringent conditions to a nucleic acid molecule having the nucleic acid sequence SEQ ID No. 27, SEQ ID No. 29, or SEQ ID No. 31.

16. A method according to claim 1, wherein the method is carried out as a one-pot reaction without isolation of intermediates.

17. A method according to claim 1, wherein the enzymes exist as a homogenate or lysate, lyophilisate, or spray-dried enzyme product of the corresponding cells producing them, the enzymes are modified with a water-soluble polymer, preferably polyethylene glycol, at the N-terminus, or the enzymes are immobilized in or on a solid matrix, or are part of a fusion protein.

18. A method according to claim 11, wherein the NAD(P)⁺-dependent oxidoreductase for the oxidation of D-talitol to D-tagatose comprises an amino acid sequence selected from the group consisting of:
i) an amino acid sequence having an identity to SEQ ID No. 4 of at least 80%,
ii) an amino acid sequence encoded by a nucleic acid having an identity to SEQ ID No. 3 of at least 80%, and
iii) an amino acid sequence encoded by a nucleic acid that binds under stringent conditions to a nucleic acid molecule having the nucleic acid sequence SEQ ID No. 3.

19. A method according to claim 11, wherein the NAD(P)⁺-dependent oxidoreductase for the oxidation of D-talitol to D-psicose comprises an amino acid sequence selected from the group consisting of:
i) an amino acid sequence having an identity to SEQ ID No. 2, SEQ ID No. 6, SEQ ID No. 8, SEQ ID No. 10, SEQ ID No. 12, SEQ ID No. 14, SEQ ID No. 16, SEQ ID No. 18, or SEQ ID No. 20 of at least 80%,
ii) an amino acid sequence encoded by a nucleic acid having an identity to SEQ ID No. 1, SEQ ID No. 5, SEQ ID No. 7, SEQ ID No. 9, SEQ ID No. 11, SEQ ID No. 13, SEQ ID No. 15, SEQ ID No. 17, or SEQ ID No. 19 of at least 80%, and
iii) an amino acid sequence encoded by a nucleic acid that binds under stringent conditions to a nucleic acid molecule having the nucleic acid sequence SEQ ID No. 1, SEQ ID No. 5, SEQ ID No. 7, SEQ ID No. 9, SEQ ID No. 11, SEQ ID No. 13, SEQ ID No. 15, SEQ ID No. 17, or SEQ ID No. 19.

20. A method for the production of D-tagatose, comprising the step of treating D-talitol with an oxidoreductase comprising an amino acid sequence selected from the group consisting of:
i) an amino acid sequence having an identity to SEQ ID No. 4 of at least 80%,
ii) an amino acid sequence encoded by a nucleic acid having an identity to SEQ ID No. 3 of at least 80%, and
iii) an amino acid sequence encoded by a nucleic acid that binds under stringent conditions to a nucleic acid molecule having the nucleic acid sequence SEQ ID No. 3.

21. (canceled)

22. A method according to claim 5, wherein the formate dehydrogenase comprises an amino acid sequence selected from the group consisting of:
i) an amino acid sequence having an identity to SEQ ID No. 26 of at least 80%,
ii) an amino acid sequence encoded by a nucleic acid having an identity to SEQ ID No. 25 of at least 80%, and
iii) an amino acid sequence encoded by a nucleic acid that binds under stringent conditions to a nucleic acid molecule having the nucleic acid sequence SEQ ID No. 25.

23. A method according to claim 11, wherein the formate dehydrogenase comprises an amino acid sequence selected from the group consisting of:
i) an amino acid sequence having an identity to SEQ ID No. 26 of at least 80%,
ii) an amino acid sequence encoded by a nucleic acid having an identity to SEQ ID No. 25 of at least 80%, and
iii) an amino acid sequence encoded by a nucleic acid that binds under stringent conditions to a nucleic acid molecule having the nucleic acid sequence SEQ ID No. 25.
